(19) United States
(12) Patent Application Publication
(10) Pub. No.: US 2026/0114823 A1
(43) Pub. Date: Apr. 30, 2026

(54) X-RAY CT APPARATUS, INFORMATION PROCESSING APPARATUS, INFORMATION PROCESSING METHOD, AND STORAGE MEDIUM

(71) Applicant: CANON MEDICAL SYSTEMS CORPORATION, Otawara-shi (JP)

(72) Inventors: Gakuto AOYAMA, Kawasaki (JP); Yoshinori HIRANO, Nasushiobara (JP)

(73) Assignee: CANON MEDICAL SYSTEMS CORPORATION, Otawara-shi (JP)

(21) Appl. No.: 19/347,969

(22) Filed: Oct. 2, 2025

(30) Foreign Application Priority Data
Oct. 25, 2024 (JP) 2024-188223

(51) Int. Cl. A61B 6/03; A61B 6/00; A61B 6/42

(52) U.S. Cl. CPC A61B 6/03; A61B 6/4208; A61B 6/5205

(57) ABSTRACT

An X-ray CT apparatus according to an embodiment includes an X-ray tube, an X-ray detector, at least one memory, and at least one piece of processing circuitry. The processing circuitry acquires a first prompt including medical information and a second prompt similar to the first prompt. The processing circuitry inputs the first prompt to a generative model and acquires first generated information corresponding to the first prompt from the generative model. The processing circuitry inputs the second prompt into the generative model and acquires second generated information corresponding to the second prompt from the generative model. The processing circuitry evaluates relevance between the first generated information and the second generated information. An embodiment discloses an information processing apparatus, an information processing method, and a storage medium.

### CROSS-REFERENCE TO RELATED APPLICATIONS

This application is based upon and claims the benefit of priority from Japanese Patent Application No. 2024-188223, filed on October 25, 2024; the entire contents of all of which are incorporated herein by reference.

### FIELD

Embodiments disclosed herein and in the drawings relate generally to an X-ray CT apparatus, an information processing apparatus, an information processing method, and a storage medium.

### BACKGROUND

In recent years, the use of generated information that is generated by generative artificial intelligence (AI) has been increasing in the medical field. Such a technology can perform easy extraction of abnormal areas, generation of image data, and the like by inputting prompts into a generative model. However, the information generated by such a generative model is not always reliable.

### BRIEF DESCRIPTION OF THE DRAWINGS

FIG. 1 is a diagram illustrating an example of the overall configuration of an information processing system according to a first embodiment;

FIG. 2 is a diagram illustrating an example of a first prompt and first generated information according to the first embodiment;

FIG. 3 is a diagram illustrating an example of a second prompt and second generated information according to the first embodiment;

FIG. 4 is a diagram illustrating an example of the first generated information according to the first embodiment;

FIG. 5 is a diagram illustrating an example of an evaluation criterion for the relevance between the first generated information and the second generated information according to the first embodiment;

FIG. 6 is a diagram illustrating an example of a case in which the locations and area ranges of lesions are identical in the first generated information and the second generated information according to the first embodiment;

FIG. 7 is a diagram illustrating an example of a case in which the locations of lesions are different and area ranges of the lesions overlap in the first generated information and the second generated information according to the first embodiment;

FIG. 8 is a diagram illustrating an example of a case in which the locations of lesions are different and area ranges of the lesions do not overlap in the first generated information and the second generated information according to the first embodiment;

FIG. 9 is a diagram illustrating an example of a case in which a lesion is detected in the first generated information and no lesion is detected in the second generated information according to the first embodiment;

FIG. 10 is a diagram illustrating an example of a display mode of evaluation results according to the first embodiment;

FIG. 11 is a flowchart showing an example of the procedure of an evaluation process according to the first embodiment;

FIG. 12 is a flowchart showing an example of the procedure of an evaluation process according to a third embodiment;

FIG. 13 is a diagram illustrating an example of a first prompt and first generated information according to a fifth embodiment;

FIG. 14 is a diagram illustrating an example of a second prompt and second generated information according to the fifth embodiment;

FIG. 15 is a diagram illustrating an example of a first prompt and first generated information according to a sixth embodiment;

FIG. 16 is a diagram illustrating an example of a second prompt and second generated information according to the sixth embodiment;

FIG. 17 is a diagram illustrating an example of a first prompt and first generated information according to a seventh embodiment;

FIG. 18 is a diagram illustrating an example of a second prompt and second generated information according to the seventh embodiment;

FIG. 19 is a diagram illustrating another example of a first prompt and first generated information according to the seventh embodiment;

FIG. 20 is a diagram illustrating another example of a second prompt and second generated information according to the seventh embodiment; and

FIG. 21 is a diagram illustrating an example of a hardware configuration of an X-ray CT apparatus according to an eighth embodiment.

### DETAILED DESCRIPTION

Embodiments of an X-ray CT apparatus, an information processing apparatus, an information processing method, and a storage medium will be described below in detail with reference to the drawings.

An X-ray CT apparatus according to an embodiment includes an X-ray tube, an X-ray detector, at least one memory, and at least one piece of processing circuitry connected to the memory. The X-ray tube irradiates a subject with X-rays. The X-ray detector detects the X-rays emitted from the X-ray tube. The processing circuitry acquires a first prompt including medical information and a second prompt similar to the first prompt. The processing circuitry inputs the first prompt to a generative model and acquires first generated information corresponding to the first prompt from the generative model. The processing circuitry inputs the second prompt into the generative model and acquires second generated information corresponding to the second prompt from the generative model. The processing circuitry evaluates relevance between the first generated information and the second generated information.

An information processing apparatus according to an embodiment includes at least one memory and at least one piece of processing circuitry connected to the memory. The processing circuitry acquires a first prompt including medical information and a second prompt similar to the first prompt. The processing circuitry inputs the first prompt to a generative model and acquires first generated information corresponding to the first prompt from the generative model. The processing circuitry inputs the second prompt into the generative model and acquires second generated information corresponding to the second prompt from the generative model. The processing circuitry evaluates relevance between the first generated information and the second generated information.

### First Embodiment

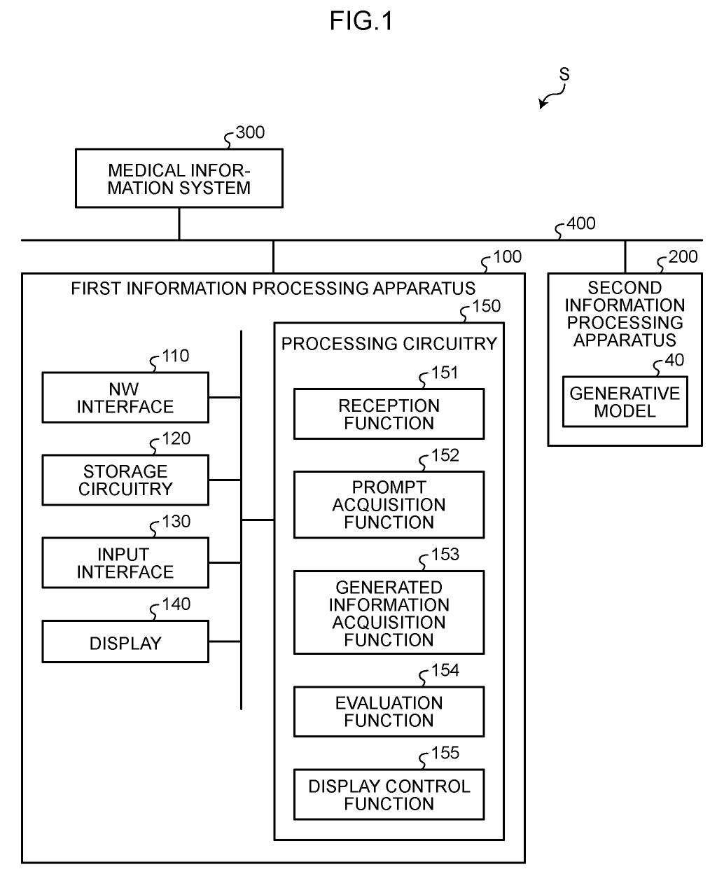

FIG. 1 is a diagram illustrating an example of the overall configuration of an information processing system S according to a first embodiment. As illustrated in FIG. 1, the information processing system S includes, as an example, a first information processing apparatus 100, a second information processing apparatus 200, and a medical information system 300. The information processing system S is provided in a medical institution such as a hospital. The first information processing apparatus 100, the second information processing apparatus 200, and the medical information system 300 are communicatively connected via a network 400 such as an in-hospital local area network (LAN).

The medical information system 300 is a system for storing and managing medical information and includes, for example, one or more servers or personal computers (PCs). The medical information stored in the medical information system 300 is, for example, electronic medical records, medical treatment information, interview results, inspection data, medical image data, diagnostic results, and the like of a patient. The medical information system 300 may be, for example, an electronic medical record system, a hospital information system (HIS), a laboratory information system (LIS), a radiology information system (RIS), a medical image storage device, or the like. The medical information system 300 may include one or more of the above systems, devices, and the like.

The first information processing apparatus 100 and the second information processing apparatus 200 are, for example, computers such as servers or PCs.

The second information processing apparatus 200 includes a learned model (hereinafter, referred to as a generative model 40) of generative artificial intelligence (AI). The second information processing apparatus 200 includes, for example, storage circuitry and a processor. The generative model 40 is stored, for example, in the storage circuitry in the second information processing apparatus 200 and operated by a processor. The configuration of the second information processing apparatus 200 is not particularly limited, and any known configuration can be adopted.

When a prompt is received, the generative model 40 generates information corresponding to the prompt and outputs the generated information. The generative model 40 is, for example, a multimodal model (MMM), a large language model (LLM) that can handle multiple types of data such as image data, or the like, and is an integrated AI model that can process multiple types of modalities (data types), such as text, images, audio, and numerical values, at once.

The generative model 40 of the present embodiment, for example, is assumed to have been trained by associating medical image data with abnormal areas included in the medical image data. The abnormal areas are, for example, lesions. The medical image data is, for example, medical image data in a format compliant with digital imaging and communications in medicine (DICOM).

The information generated by the generative model 40 is referred to as generated information. The generated information includes, for example, image data. The generated information may include text as well as image data. Depending on the content of the prompt, the generated information may include no image data. In the present embodiment, since the information processing system S is used in the medical field, the prompt and the generated information include, for example, information on a patient. The information on a patient is, for example, medical information such as inspection results, interview results, and findings by a physician. The information on a patient may also be information on patient's gender, age, and physique. The prompt and the generated information may also include medical image data. The medical image data is also referred to as medical-treatment image data.

The first information processing apparatus 100 is operated by a user, and provides the user with output results of the generative model 40 stored in the second information processing apparatus 200. The user of the first information processing apparatus 100 is, for example, a physician or other medical professionals. For example, a physician may use the generative model 40 via the first information processing apparatus 100 in order to obtain information to be referred for patient diagnosis. The first information processing apparatus 100 is an example of an information processing apparatus in the present embodiment.

The first information processing apparatus 100 includes a network (NW) interface 110, storage circuitry 120, an input interface 130, a display 140, and processing circuitry 150.

The NW interface 110 is connected to the processing circuitry 150. The NW interface 110 controls the transmission and communication of various data performed among the first information processing apparatus 100, the second information processing apparatus 200, and the medical information system 300. The NW interface 110 is implemented by a network card, a network adapter, a network interface controller (NIC), or the like.

The storage circuitry 120 stores in advance various information used by the processing circuitry 150. The storage circuitry 120 also stores various computer programs. The storage circuitry 120 is a non-volatile storage device storing various information, such as a hard disk drive (HDD), a solid state drive (SSD), or an integrated circuit storage device. In addition to the HDD, the SSD, or the like, the storage circuitry 120 may also be a drive device that reads and writes various information from/to a portable storage medium such as a compact disc (CD), a digital versatile disc (DVD), or a flash memory, or a semiconductor memory element such as a random access memory (RAM). The storage circuitry 120 is an example of a storage unit.

The input interface 130 is implemented by a mouse, a keyboard, a pen tablet that combines a touch pen and a tablet to receive user's operations, a trackball, a switch button, a touchpad that performs input operations by touching an operation surface, a touchscreen that integrates a display screen and a touchpad, non-contact input circuitry using an optical sensor, audio input circuitry, or the like. The input interface 130 may include a plurality of devices that receive user's operations. The input interface 130 is connected to the processing circuitry 150, converts input operations received from the user into electrical signals, and outputs the electrical signals to the processing circuitry 150. In the present specification, the input interface 130 is not limited only to those with physical operating components such as a mouse or a keyboard. For example, electrical signal processing circuitry that receives electrical signals corresponding to input operations from an external input device provided separately from the device and outputs the electrical signals to the processing circuitry 150 is also included in an example of the input interface 130.

The display 140 displays various information under the control of the processing circuitry 150. For example, the display 140 outputs a screen including the generated information generated by the generative model 40, a graphical user interface (GUI) for receiving various operations from the user, or the like. The display 140 is specifically a liquid crystal display, an organic EL display, a cathode ray tube (CRT) display, or the like. The input interface 130 and the display 140 may be integrated. For example, the input interface 130 and the display 140 may be implemented by a touch panel.

The processing circuitry 150 is a processor that reads computer programs from the storage circuitry 120 and executes the computer programs to implement functions corresponding to the executed computer programs. The processing circuitry 150 of the present embodiment includes a reception function 151, a prompt acquisition function 152, a generated information acquisition function 153, an evaluation function 154, and a display control function 155. The reception function 151 is an example of a reception unit. The prompt acquisition function 152 is an example of a prompt acquisition unit. The generated information acquisition function 153 is an example of a generated information acquisition unit. The evaluation function 154 is an example of an evaluation unit. The display control function 155 is an example of an output unit or a display control unit.

For example, the processing functions of the reception function 151, the prompt acquisition function 152, the generated information acquisition function 153, the evaluation function 154, and the display control function 155, which are components of the processing circuitry 150, are stored in the storage circuitry 120 in the form of computer programs executable by a computer. The processing circuitry 150 is a processor. For example, the processing circuitry 150 reads the computer programs from the storage circuitry 120 and executes the computer programs to implement the functions corresponding to the executed computer programs. In other words, the processing circuitry 150 in the state of having read each computer program has each of the functions shown within the processing circuitry 150 in FIG. 1. In FIG. 1, the processing functions performed by the reception function 151, the prompt acquisition function 152, the generated information acquisition function 153, the evaluation function 154, and the display control function 155 are described as being implemented by a single processor; however, the processing circuitry 150 may be configured by combining two or more independent processors and the respective processors may implement functions by executing computer programs. In FIG. 1, single storage circuitry 120 is described as storing a computer program corresponding to each processing function; however, two or more pieces of storage circuitry may be dispersively disposed and the processing circuitry 150 may be configured to read a corresponding computer program from the individual pieces of storage circuitry.

The above explanation describes an example in which the "processor" reads a computer program corresponding to each function from the storage circuitry 120 and executes the computer program; however, the embodiment is not limited thereto. The term "processor" means, for example, circuitry such as a central processing unit (CPU), a graphics processing unit (GPU), an application specific integrated circuit (ASIC), or a programmable logic device (for example, a simple programmable logic device (SPLD), a complex programmable logic device (CPLD), and a field programmable gate array (FPGA)). When the processor is, for example, a CPU, the processor reads and executes the computer program stored in the storage circuitry 120 to implement the function. On the other hand, when the processor is an ASIC, instead of storing the computer program in the storage circuitry 120, a corresponding function is directly incorporated into the processor's circuitry as logic circuitry. Each processor of the present embodiment is not limited to the case in which each processor is configured as single circuitry, but may also be configured as a single processor by combining two or more pieces independent circuitry to implement functions thereof. Moreover, the components in FIG. 1 may be integrated into a single processor to implement functions thereof.

The reception function 151 receives various user's operations via the input interface 130. For example, the reception function 151 receives user's operations instructing the start of a process.

The prompt acquisition function 152 acquires prompts to be input to the generative model 40. More specifically, the prompt acquisition function 152 acquires a first prompt and a second prompt input by the user.

The first prompt is a prompt for the user to obtain desired information. For example, the user inputs the first prompt in order to obtain information required for diagnosing a patient from the generative model 40. The second prompt is a prompt used for evaluating the reliability of first generated information generated by a first prompt 501. Therefore, the second prompt itself need not be the content that the user desires for diagnosing the patient. The second prompt is also similar to the first prompt.

Since the information processing system S of the present embodiment is used, for example, in the medical field, the first and second prompts input to the generative model 40 include medical information. The medical information is, for example, electronic medical records, medical treatment information, interview results, inspection data, medical image data, diagnostic results, and the like of a subject (patient), but is not limited thereto.

More specifically, the first and second prompts to be input to the generative model 40 each include at least one of medical image data or medical-related text data.

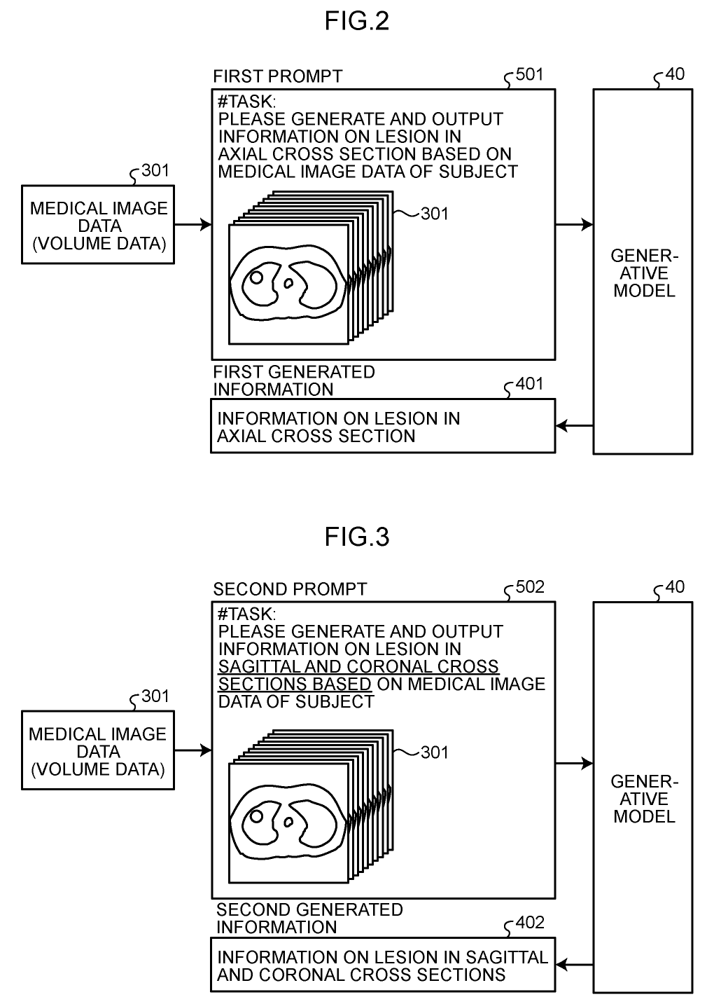

FIG. 2 is a diagram illustrating an example of the first prompt 501 and first generated information 401 according to the first embodiment. The first prompt 501 includes, for example, an instruction to cause the generative model 40 to generate information on a first abnormal area in a first cross section of medical image data 301 as the first generated information 401. The instruction is included in the first prompt 501, for example, as text data with a tag "#task".

As a specific example, as illustrated in FIG. 2, the first prompt 501 includes, for example, text data such as "Please generate and output information on a lesion in an axial cross section based on medical image data of a subject" and the medical image data 301 corresponding to the "medical image data of the subject" of the text data. The text data is input, for example, by the user. The axial cross section is an example of the first cross section. The description of "lesion" in the first prompt 501 may be an "abnormal area" or other description.

The medical image data 301 is, for example, three-dimensional volume data of X-ray computed tomography (CT) image data or magnetic resonance image data. The medical image data 301 may be X-ray image data, ultrasound image data, or the like. The medical image data 301 is, for example, image data acquired from the medical information system 300.

A mode in which the text data of the prompt includes the medical image data 301 is not particularly limited, but for example, the path of a storage destination of the medical image data 301 may be specified in the text data of the prompt. For example, the medical image data 301 can be included in the prompt in a mode in which the generative model 40 is retrievable, such as retrieval-augmented generation (RAG). In addition to the medical image data 301, the storage destination of various medical information stored in the medical information system 300 may be listed in the text data of the first prompt 501.

The storage destination of the medical information referenced from the text data of the prompt may be a path indicating a storage location within the medical information system 300 or a path indicating a storage location within the storage circuitry 120 of the first information processing apparatus 100. The medical information may also be directly described as text data in the first prompt 501.

The content of the first prompt 501 is not limited to the example illustrated in FIG. 2. For example, a lesion is an example of an abnormal area, and a "lesion" in the text data may be replaced with the more comprehensive concept of "abnormal area". Alternatively, other specific objects may be specified in the text data, such as "tumor" or "polyp". Other commands for the generative model 40 may also be included in the text data depending on the user's application. In the present embodiment, a lesion is described below as an example of an abnormal area.

FIG. 3 is a diagram illustrating an example of a second prompt 502 and second generated information 402 according to the first embodiment.

As described above, the second prompt 502 is a prompt similar to the first prompt 501. More specifically, the first prompt 501 and the second prompt 502 differ at least in part and are identical except for the difference. The difference between the first prompt 501 and the second prompt 502 relates to a wording or a display mode, and the first prompt 501 and the second prompt 502 are medically synonymous.

The second prompt 502 includes, for example, an instruction to cause the generative model 40 to generate information on a second abnormal area in a second cross section of the medical image data 301 as the second generated information 402. The second cross section is a cross section different from the first cross section. In the example illustrated in FIG. 3, the second prompt 502 includes, for example, text data such as "Please generate and output information on a lesion in sagittal and coronal cross sections based on medical image data of a subject" and the medical image data 301 corresponding to the "medical image data of the subject" of the text data. The sagittal and coronal cross sections are examples of the second cross section. In the example illustrated in FIG. 3, the second prompt 502 includes the specification of two types of cross sections: sagittal and coronal cross sections, but only one of the two types may be specified.

The medical image data 301 included in the second prompt 502 is the same three-dimensional volume data as the medical image data 301 included in the first prompt 501. The text data of the second prompt 502 differs from the first prompt 501 in the type of cross section for which information on a lesion is generated. Specifically, the first prompt 501 specifies an axial cross section, while the second prompt 502 specifies sagittal and coronal cross sections. In other words, the first prompt 501 and the second prompt 502 differ in the type of cross section, but the rest of the prompts is identical.

In addition, since the medical image data 301 is three-dimensional volume data, a difference in the display mode occurs depending on in which direction a cross section is cut is displayed. However, since the difference is related to the display mode and the medical image data 301 being the target of lesion specification is identical, the first prompt 501 and the second prompt 502 are medically synonymous.

The content of the first and second prompts 501 and 502 illustrated in FIGS. 2 and 3 is an example and is not limited thereto.

Returning to FIG. 1, the generated information acquisition function 153 inputs the first and second prompts 501 and 502 acquired by the prompt acquisition function 152 to the generative model 40 of the second information processing apparatus 200 via the network 400 and the NW interface 110, thereby acquiring the first and second generated information from the generative model 40. The first generated information corresponds to the first prompt 501 and the second generated information corresponds to the second prompt 502.

In the example illustrated in FIG. 2 above, when the first prompt 501 is received, the generative model 40 outputs the first generated information 401 based on the instruction of the first prompt 501. Specifically, when the first prompt 501 is input, the generative model 40 detects lesions in the axial cross section of the medical image data 301, generates information on the detected lesions as the first generated information 401, and outputs the first generated information 401. The information on the lesions includes the location information of the lesions. The location information of the lesions is, for example, information indicating the locations, area ranges, and number of lesions. The generated information acquisition function 153 stores the first generated information 401 and the second generated information 402 to be described below in the storage circuitry 120.

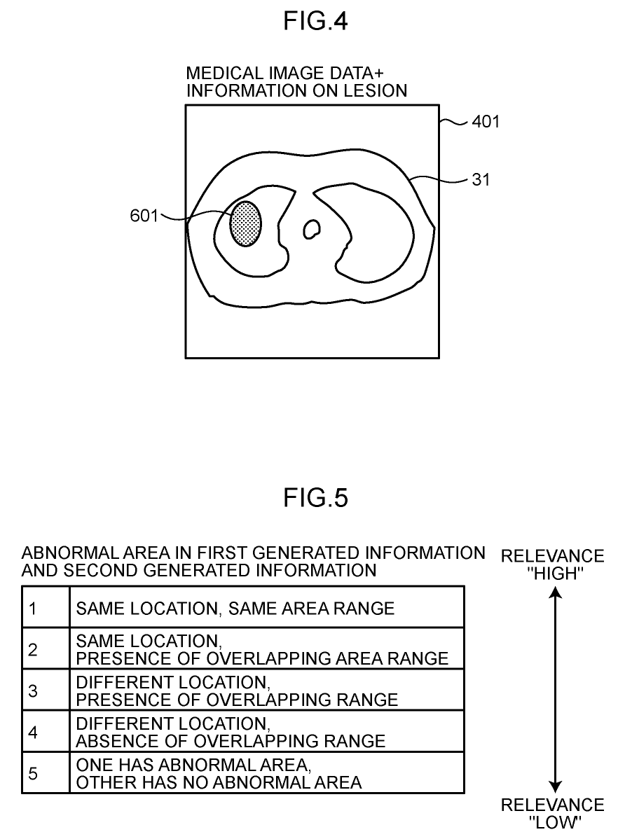

FIG. 4 is a diagram illustrating an example of the first generated information 401 according to the first embodiment. The first generated information 401 is, for example, image data in which first abnormal area information 601 indicating the locations, area ranges, and number of lesions (abnormal areas) is superimposed on two-dimensional image data 31 indicating the axial cross section of the medical image data 301 included in the first prompt 501, as illustrated in FIG. 4. More specifically, the first abnormal area information 601 indicates the locations and the area ranges of the lesions (abnormal areas). In FIG. 4, the number of lesions (abnormal areas) specified is one, but when two or more lesions (abnormal areas) are specified, the number of first abnormal area information 601 on the two-dimensional image data 31 is plural. In the present embodiment, the first generated information 401 is data to which the first abnormal area information 601 is added to the medical image data 301 input to the generative model 40 as an analysis target. Therefore, the medical image data 301 in the first generated information 401 output from the generative model 40 is identical to the medical image data 301 input to the generative model 40 as an analysis target. In this case, the first abnormal area information 601 added to the medical image data 301 by the generative model 40 may be referred to as the first generated information in the medical image data 301.

The generative model 40 specifies a lesion from the medical image data 301, and generates, as the two-dimensional image data 31, a cross sectional position where the lesion is most significantly displayed in the axial cross section specified by the first prompt 501.

The first abnormal area information 601 is an example of information on the first abnormal area included in the first generated information 401 generated by the generative model 40. The display mode of the first abnormal area information 601 is not limited to the example illustrated in FIG. 4.

On the basis of the instruction included in the second prompt 502, the generative model 40 generates, as the second generated information 402, lesion image data indicating the location and area range of the lesion in the sagittal and coronal cross sections of the medical image data 301, and outputs the second generated information 402. In the present embodiment, the second generated information 402 is data to which the second abnormal area information is added to the medical image data 301 input to the generative model 40 as an analysis target. Therefore, the medical image data 301 in the second generated information 402 output from the generative model 40 is identical to the medical image data 301 input to the generative model 40 as an analysis target. In this case, the second abnormal area information added to the medical image data 301 by the generative model 40 may be referred to as the second generated information in the medical image data 301. The second abnormal area information indicates the location and the area range of the lesion (abnormal area) in the same way as the first abnormal area information 601.

Returning to FIG. 1, the evaluation function 154 evaluates the relevance between the first generated information 401 and the second generated information 402. The relevance between the first generated information 401 and the second generated information 402 in the present embodiment is expressed, for example, by the degree of similarity between the first generated information 401 and the second generated information 402. The higher the degree of similarity between the first generated information 401 and the second generated information 402, the evaluation function 154 evaluates that the reliability of the generative model 40 is higher. In other words, the evaluation function 154 indirectly evaluates the reliability of the generative model 40 by evaluating the relevance between the first generated information 401 and the second generated information 402. Evaluating the reliability of the generative model 40 also means evaluating the reliability of the generated information generated by the generative model 40.

More specifically, the evaluation function 154 evaluates the relevance between the first generated information 401 and the second generated information 402 on the basis of the locations, area ranges, and number of the first abnormal area included in the first generated information 401 and the second abnormal area included in the second generated information 402.

FIG. 5 is a diagram illustrating an example of an evaluation criterion for the relevance between the first generated information 401 and the second generated information 402 according to the first embodiment. As illustrated in FIG. 5, the evaluation function 154 evaluates that as the locations and area ranges of the lesions in the axial cross section in the first generated information 401 and the sagittal and coronal cross sections in the second generated information 402 are closer to be identical, the relevance between the first generated information 401 and the second generated information 402 is higher. The location of the lesion in the comparison of the first generated information 401 and the second generated information 402 is based on the center location or center of gravity location of the lesion, for example. Since the medical image data 301 to be analyzed in the present embodiment is three-dimensional volume data, the location of the lesion is a three-dimensional location in the entire medical image data 301. Therefore, the location and the area range of the lesion are indicated by three-dimensional coordinates in the medical image data 301.

As described above, the first prompt 501 and the second prompt 502 differ only in the type of cross section to be specified, but instructions regarding the medical image data 301 to be analyzed and an object to be specified from the medical image data 301 are identical. Therefore, the locations, area ranges, and number of lesions specified in the first generated information 401 and the second generated information 402 are preferably identical.

However, depending on the amount, content, and the like of prior training of the generative model 40, the accuracy of lesion specification may vary depending on the type of cross section to be analyzed. For example, when training data in the prior training of the generative model 40 is biased toward lesion specification results in the axial cross section, the accuracy of lesion specification in the sagittal and coronal cross sections may be inferior to that in the axial cross section. In such a case, the type of cross section specified in the prompt may cause fluctuations in the locations, area ranges, and number of lesions in the generated information of the generative model 40. The accuracy of the first generated information 401 may also be low when the medical information and specific instructions included in the first prompt 501 and the second prompts 502 are missing, even though the generative model 40 have been sufficiently trained in advance. In this case, the output of the generative model 40 is more likely to fluctuate, and the difference between the first generated information 401 and the second generated information 402 may be increased.

By evaluating the relevance between the first generated information 401 and the second generated information 402, the evaluation function 154 evaluates that the reliability of the generative model 40 with small fluctuations due to the type of cross section specified in such prompts is high and the reliability of the generative model 40 with large fluctuations is low.

In the example illustrated in FIG. 5, the evaluation function 154 evaluates that the relevance between the first generated information 401 and the second generated information 402 is the highest when the locations and area ranges of the lesions (abnormal areas) are identical in the axial cross section in the first generated information 401 and the sagittal and coronal cross sections in the second generated information 402. In other words, in this case, the evaluation function 154 evaluates that the reliability of the generative model 40 is the highest.

When, as in the example illustrated in FIG. 3, the second generated information 402 instructs the generation of information on lesions in two types of cross sections, that is, the sagittal cross section and the coronal cross section, the case, in which the location and area range of both lesions on the sagittal cross section and on the coronal cross section in the second generated information 402 are identical to the location and area range of the lesion on the axial cross section in the first generated information 401, corresponds to a first pattern illustrated in FIG. 5.

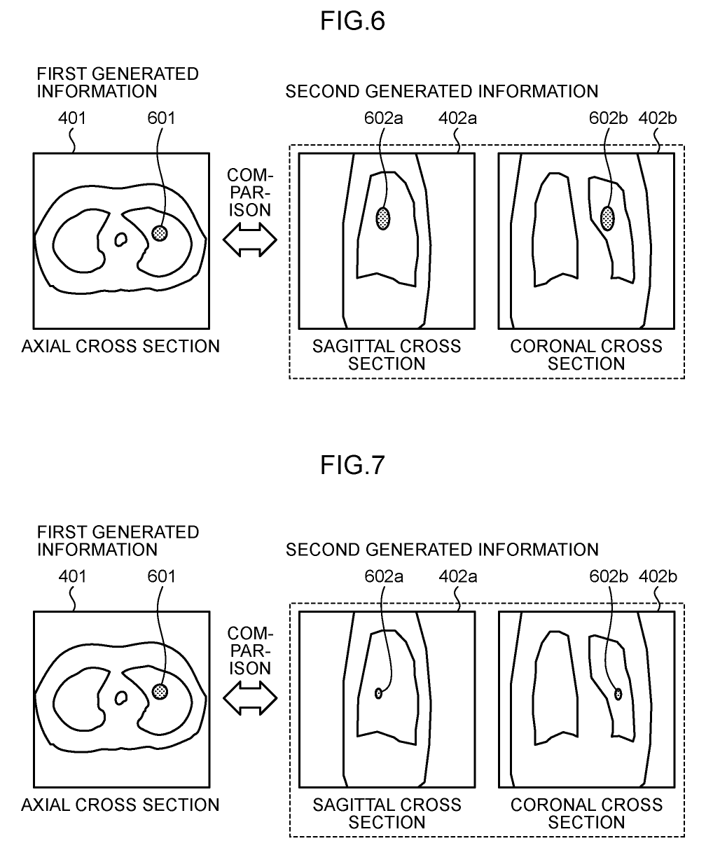

FIG. 6 is a diagram illustrating an example of a case in which the locations and area ranges of lesions are identical in the first generated information 401 and the second generated information 402 according to the first embodiment. When the locations, area ranges, and number of lesions indicated by the first abnormal area information 601 in the first generated information 401 and the locations, area ranges, and number of lesions indicated by second abnormal area information 602a and 602b in the second generated information 402 are identical, since the first pattern illustrated in FIG. 5 is satisfied, the evaluation function 154 evaluates that the reliability of the generative model 40 is the highest.

In the example illustrated in FIG. 5, when the locations of the lesions (abnormal areas) are identical and the area ranges of the lesions (abnormal areas) overlap in the axial cross section in the first generated information 401 and the sagittal and coronal cross sections in the second generated information 402, the evaluation function 154 evaluates that the relevance between the first generated information 401 and the second generated information 402 is the second highest.

In the example illustrated in FIG. 5, when the locations of the lesions (abnormal areas) are different and the area ranges of the lesions (abnormal areas) overlap in the axial cross section in the first generated information 401 and the sagittal and coronal cross sections in the second generated information 402, the evaluation function 154 evaluates that the relevance between the first generated information 401 and the second generated information 402 is the third highest.

The number of lesions (abnormal areas) detected in the first generated information 401 may differ from the number of lesions (abnormal areas) detected in the second generated information 402. In this case, when an overlapping range is present between the area range of plural lesions (abnormal areas) detected in the first generated information 401 and the area range of plural lesions (abnormal areas) detected in the second generated information 402, the third pattern illustrated in FIG. 5 is applicable.

The evaluation function 154 may further specify the degree of relevance in more detail by evaluating the relevance higher the greater the percentage of overlap in the area ranges of the lesions (abnormal areas) when the second and third patterns illustrated in FIG. 5 are applicable.

FIG. 7 is a diagram illustrating an example of a case in which the locations of lesions are different and area ranges of the lesions overlap in the first generated information 401 and the second generated information 402 according to the first embodiment.

In the example illustrated in FIG. 7, the center of gravity location of the lesion indicated by the first abnormal area information 601 in the first generated information 401 is different from the center of gravity location of the lesion indicated by the second abnormal area information 602a and 602b in the second generated information 402. In the example illustrated in FIG. 7, the area range of the lesion indicated by the first abnormal area information 601 in the first generated information 401 encompasses the area range of the lesion indicated by the second abnormal area information 602a and 602b in the second generated information 402. Therefore, since an overlapping range is present between the area range of the lesion detected in the first generated information 401 and the area range of the lesion detected in the second generated information 402, the third pattern illustrated in FIG. 5 is applicable.

In the example illustrated in FIG. 5, when the locations of the lesions (abnormal areas) are different and the area ranges of the lesions (abnormal areas) overlap in the axial cross section in the first generated information 401 and in the sagittal and coronal cross sections in the second generated information 402, the evaluation function 154 evaluates that the relevance between the first generated information 401 and the second generated information 402 is the fourth highest.

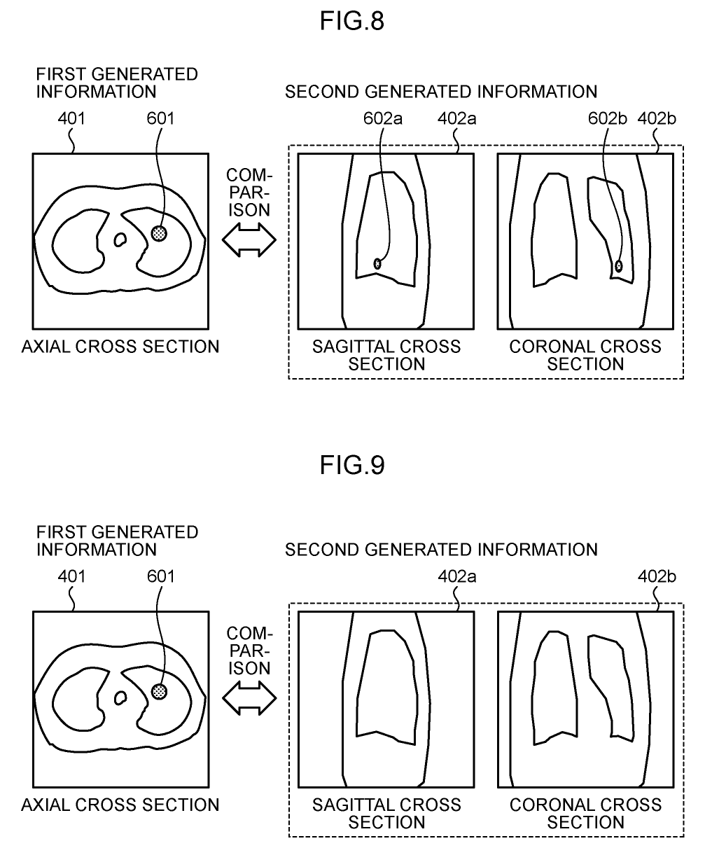

FIG. 8 is a diagram illustrating an example of a case in which the locations of lesions are different and area ranges of the lesions do not overlap in the first generated information 401 and the second generated information 402 according to the first embodiment.

In the example illustrated in FIG. 8, the center of gravity location of the lesion indicated by the first abnormal area information 601 in the first generated information 401 is different from the center of gravity location of the lesion indicated by the second abnormal area information 602a and 602b in the second generated information 402. In the example illustrated in FIG. 8, the area range of the lesion indicated by the first abnormal area information 601 in the first generated information 401 does not overlap the area range of the lesion indicated by the second abnormal area information 602a and 602b in the second generated information 402. Therefore, the example illustrated in FIG. 8 corresponds to the fourth pattern illustrated in FIG. 5.

In the fifth example illustrated in FIG. 5, when a lesion (abnormal area) is detected in either the first generated information 401 or the second generated information 402 and no lesion (abnormal area) is detected in the other, the evaluation function 154 evaluates the relevance as the lowest.

FIG. 9 is a diagram illustrating an example of a case in which a lesion is detected in the first generated information 401 and no lesion is detected in the second generated information 402 according to the first embodiment. The second generated information 402 illustrated in FIG. 9 does not include the second abnormal area information 602a and 602b. Therefore, the example illustrated in FIG. 8 corresponds to the fifth pattern illustrated in FIG. 5.

The relevance evaluation criterion illustrated in FIG. 5 is an example and is not limited thereto. For example, the evaluation function 154 may dynamically determine the evaluation criterion according to the content of the first and second prompts 501 and 502. For example, when the first and second prompts 501 and 502 include instructions to search for or generate similar images, the evaluation function 154 may evaluate that the greater the number of identical or similar image data in the image data included in the first generated information 401 and the image data included in the second generated information 402, the higher the relevance between the first generated information 401 and the second generated information 402.

In a case in which the first and second prompts 501 and 502 include instructions to output a diagnosis name, when a diagnosis name included in the first generated information 401 is identical or similar to a diagnosis name included in the second generated information 402, the evaluation function 154 may evaluate that the relevance between the first generated information 401 and the second generated information 402 is high. When the first generated information 401 and the second generated information 402 each include a plurality of diagnosis names or pieces of image data, the evaluation function 154 may evaluate that the greater the number of identical diagnosis names or image data in the first generated information 401 and the second generated information 402, the higher the relevance between the first generated information 401 and the second generated information 402.

The number of steps in the relevance evaluation is also not limited to the example illustrated in FIG. 5. For example, the evaluation function 154 may evaluate the relationship between the first generated information 401 and the second generated information 402 at two levels, with and without relevance.

Returning to FIG. 1, the display control function 155 controls the display 140 to display various information. For example, the display control function 155 controls the display 140 to display the results of the evaluation for the relevance between the first generated information 401 and the second generated information 402 by the evaluation function 154.

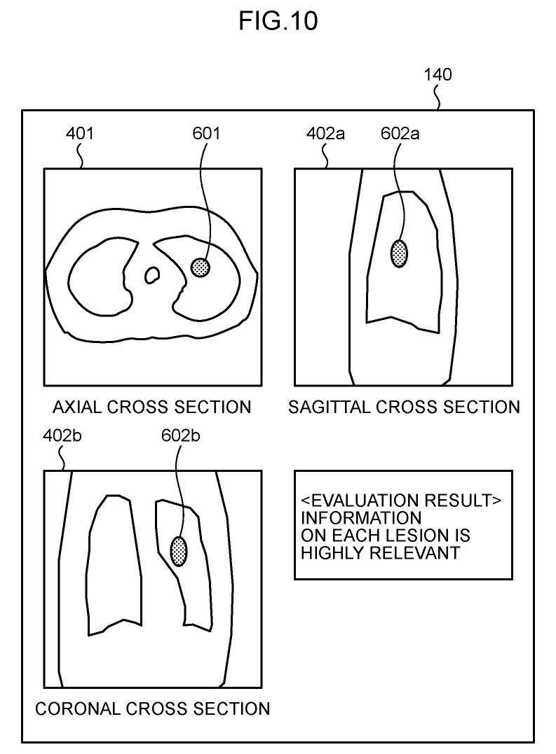

FIG. 10 is a diagram illustrating an example of a display mode of evaluation results according to the first embodiment. As illustrated in FIG. 10, the display control function 155 controls the display 140 to display, for example, the result of the evaluation for the relevance between the first generated information 401 and the second generated information 402 together with the first generated information 401 and the second generated information 402. In the example illustrated in FIG. 10, the result of the evaluation for the relevance is indicated by text such as "Information on each lesion is highly relevant". Such display of the evaluation results allows the user to recognize that the lesion detected by the generative model 40 in the axial cross section on the basis of the first prompt 501 is highly reliable.

On the other hand, when the result of the evaluation for the relevance displayed on the display 140 by the display control function 155 indicates that the evaluation for the relevance between the first generated information 401 and the second generated information 402 is low, the user can recognize that the lesion detected by the generative model 40 in the axial cross section on the basis of the first prompt 501 is less reliable. By displaying the first generated information 401 and the second generated information 402 together with the evaluation result as illustrated in FIG. 10, the user can ascertain the locations, area ranges, and number of lesions that can be estimated by taking into account fluctuations in the generative model 40.

The method of expressing the result of the evaluation for the relevance is not limited to the example illustrated in FIG. 10, and for example, the evaluation result may be indicated by graded levels indicating the degree of relevance, numerical values, or the like.

The display control function 155 may also control the result of the evaluation for the relevance to be displayed as the result of the evaluation for the reliability of the first generated information 401. For example, the display control function 155 may control the display 140 to display text such as "Output generated information is highly reliable" or "Output generated information is less reliable". In FIG. 10, both the first generated information 401 and the second generated information 402 are displayed on the display 140; however, when the user originally wanted only the first generated information 401 and the second generated information 402 was only used for evaluating the first generated information 401, the display control function 155 may control the display 140 to display only the first generated information 401 and evaluation results.

The display control function 155 may control the result of the evaluation for the relevance to be displayed together with the medical image data 301 to be analyzed included in the first prompt 501 and the second prompt 502, the first generated information 401, and the second generated information 402.

The display on the display 140 is an example of output in the present embodiment. The first information processing apparatus 100 may employ output methods other than the display on the display 140. For example, the first information processing apparatus 100 may have a transmission function, as an example of an output unit, that transmits evaluation results to other information processing apparatuses via the NW interface 110.

The display control function 155 also controls the display 140 to display a prompt input screen on which the user can input the first and second prompts 501 and 502. The prompt input screen may have a function of restricting the input of the first and second prompts 501 and 502 to a prescribed format such that the first and second prompts 501 and 502 include the medical image data 301 when the user generates the first and second prompts 501 and 502. The function of restricting the input of the first and second prompts 501 and 502 to a prescribed format is, for example, an input check function of requiring the path of a storage area where the medical image data 301 is stored to be described as a reference destination on the prompt input screen, a templated input format of the first and second prompts 501 and 502, or the like, but is not limited thereto.

The procedure of the evaluation process performed by the first information processing apparatus 100 configured as described above will be described below.

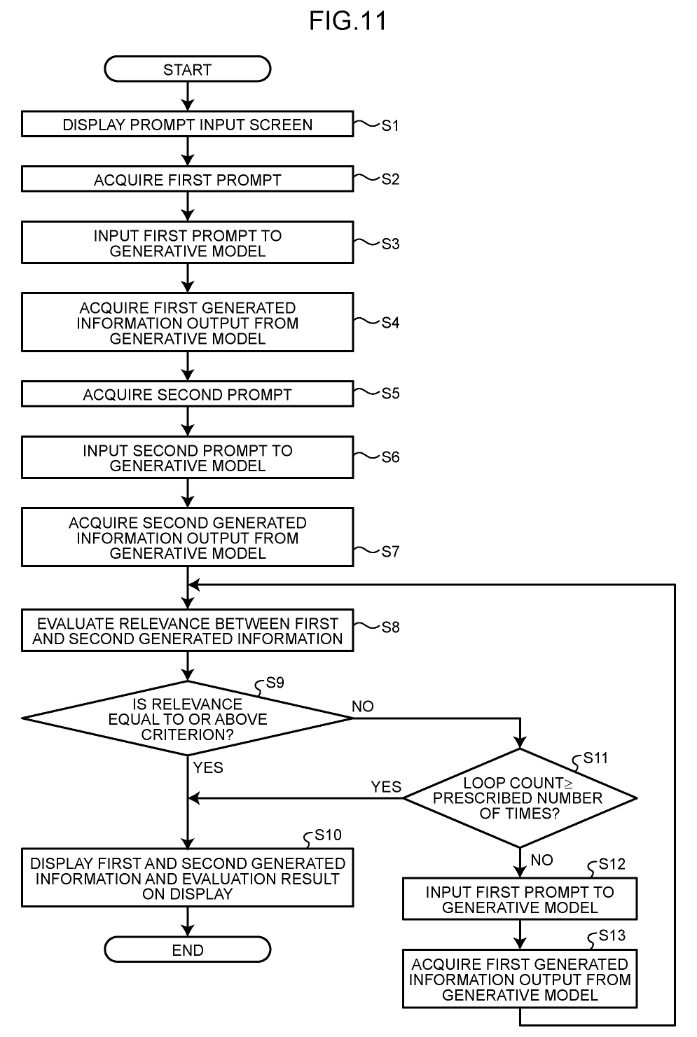

FIG. 11 is a flowchart showing an example of the procedure of the evaluation process according to the first embodiment. The process in the flowchart is performed, for example, when the reception function 151 receives an operation to open the prompt input screen from a user.

First, the display control function 155 controls the display 140 to display the prompt input screen (S1).

Subsequently, the prompt acquisition function 152 acquires the first prompt 501 input by the user (S2).

Subsequently, the generated information acquisition function 153 inputs the first prompt 501 to the generative model 40 (S3). In this case, the generative model 40 generates the first generated information 401 on the basis of the input first prompt 501.

Subsequently, the generated information acquisition function 153 acquires the first generated information 401 output from the generative model 40 (S4).

Subsequently, the prompt acquisition function 152 acquires the second prompt 502 input by the user (S5).

Subsequently, the generated information acquisition function 153 inputs the second prompt 502 to the generative model 40 (S6). In this case, the generative model 40 generates the second generated information 402 on the basis of the input second prompt 502.

Subsequently, the generated information acquisition function 153 acquires the second generated information 402 output from the generative model 40 (S7).

Subsequently, the evaluation function 154 compares the first generated information 401 and the second generated information 402 output from the generative model 40 and evaluates relevance (S8).

If the relevance in the evaluation result is equal to or above a criterion ("Yes" at S9), the evaluation function 154 fixes the evaluation result. Once the result of the evaluation for the relevance is fixed, the display control function 155 controls the display 140 to display the result of the evaluation for the relevance between the first generated information 401 and the second generated information 402 by the evaluation function 154 (S10).

For example, the criterion for fixing the evaluation for the relevance may be whether the result is equal to or above the third pattern "different locations, presence of overlapping range" from the top illustrated in FIG. 5. That is, if the evaluation result obtained at S8 indicates a higher degree of relevance between the first generated information 401 and the second generated information 402 than when "the locations of the lesions are different and area ranges of the lesions overlap in the first generated information 401 and the second generated information 402", the evaluation function 154 fixes the evaluation result. The criterion for fixing the relevance is not limited to such an example.

If the relevance in the evaluation result is below the criterion ("No" at S9), the evaluation function 154 regenerates the first generated information 401 a prescribed number of times and re-evaluates the first generated information 401 and the second generated information 402. This is because, for example, when large fluctuations occur in the output results of the generative model 40, the output results may differ even when the same prompt is input.

In this case, while the loop count has not reached the prescribed number of times ("No" at S11), the generated information acquisition function 153 inputs the first prompt 501 to the generative model 40 again (S12) and acquires the first generated information 401 output from the generative model 40 (S13). The prescribed number of times being an upper limit of the loop count is, for example, three, but is not limited thereto.

Returning to S8, the evaluation function 154 compares the regenerated first generated information 401 with the second generated information 402 and evaluates relevance.

Subsequently, the procedure proceeds to the process at S9, and if the relevance between the regenerated first generated information 401 and the second generated information 402 in the evaluation result is equal to or above the criterion, the evaluation function 154 fixes the evaluation result. The evaluation function 154 may take into account the loop count in the evaluation until the relevance is equal to or above the criterion. For example, the evaluation function 154 may subtract the evaluation for the relevance the greater the loop count until the relevance is equal to or above the criterion.

If the loop count has reached the prescribed number of times ("Yes" at S11), the evaluation function 154 fixes the latest evaluation results. Subsequently, the procedure proceeds to the process at S10, and the display control function 155 controls the display 140 to display the results of the evaluation for the relevance between the first generated information 401 and the second generated information 402 by the evaluation function 154. The procedure of the flowchart ends.

The processes at S9 and S11 to S13 are not required, and the evaluation function 154 may fix the relevance evaluation by a single relevance evaluation. In the retry processes at S11 to S13, not only the first generated information 401 but also the second generated information 402 may be regenerated.

In this way, the first information processing apparatus 100 of the present embodiment evaluates the relevance between the first generated information 401 obtained by inputting the first prompt 501 to the generative model 40 and the second generated information 402 obtained by inputting the second prompt 502 similar to the first prompt 501 to the generative model 40. That is, according to the first information processing apparatus 100 of the present embodiment, the reliability of the first generated information 401 obtained by the first prompt 501 can be verified by comparing the first generated information 401 with the second generated information 402 obtained by the second prompt 502 similar to the first prompt 501. Therefore, according to the first information processing apparatus 100 of the present embodiment, the reliability of the first generated information 401 generated by the generative model 40 can be evaluated.

The first information processing apparatus 100 of the present embodiment also outputs the results of the evaluation for the relevance between the first generated information 401 and the second generated information 402. Therefore, according to the first information processing apparatus 100 of the present embodiment, a user can ascertain the reliability of the first generated information 401.

The first information processing apparatus 100 of the present embodiment also outputs the results of the evaluation for the relevance between the first generated information 401 and the second generated information 402 together with the first generated information 401 and the second generated information 402. Therefore, according to the first information processing apparatus 100 of the present embodiment, a user can confirm the difference between the first generated information 401 and the second generated information 402 together with the evaluation results.

The first information processing apparatus 100 of the present embodiment may also output the results of the evaluation for the relevance between the first generated information 401 and the second generated information 402 together with the medical image data 301 to be analyzed included in the first prompt 501 and the second prompt 502, the first generated information 401 in the medical image data 301, and the second generated information 402 in the medical image data 301. With such a configuration, when the first prompt 501 and the second prompt 502 include an instruction to analyze the medical image data 301 as in the present embodiment, since a user can easily confirm the medical image data 301 before being processed by the generative model 40 together with the first generated information 401 and the second generated information 402, the convenience of the user in the confirmation process is improved.

The first prompt 501 and the second prompt 502 in the present embodiment include at least one of medical image data or medical-related text data. The first prompt 501 and the second prompt 502 differ at least in part and are identical except for the difference. The first information processing apparatus 100 of the present embodiment evaluates the degree of the relevance between the first generated information 401 and the second generated information 402 by the degree of similarity between the first generated information 401 and the second generated information 402. That is, the first information processing apparatus 100 of the present embodiment evaluates the degree of the relevance between the first generated information 401 and the second generated information 402 by the degree of similarity between the first generated information 401 and the second generated information 402 obtained by the first prompt 501 and the second prompt 502 similar to each other. Therefore, the first information processing apparatus 100 of the present embodiment can verify whether the generative model 40 is in a stable state with little fluctuations caused by superficial differences in prompts, through appropriate prior learning.

### Second Embodiment

In the first embodiment described above, both the first prompt 501 and the second prompt 502 are input by a user. However, in the second embodiment, the first information processing apparatus 100 automatically generates some or all of the second prompt 502.

The information processing system S of the present embodiment includes, for example, the first information processing apparatus 100, the second information processing apparatus 200, and the medical information system 300, as in the first embodiment described with reference to FIG. 1. The first information processing apparatus 100 of the present embodiment includes the NW interface 110, the storage circuitry 120, the input interface 130, the display 140, and the processing circuitry 150, as in the first embodiment.

The processing circuitry 150 of the present embodiment includes the reception function 151, the prompt acquisition function 152, the generated information acquisition function 153, the evaluation function 154, and the display control function 155, as in the first embodiment.

The reception function 151, the generated information acquisition function 153, the evaluation function 154, and the display control function 155 have the same functions as in the first embodiment.

In addition to the functions of the first embodiment, the prompt acquisition function 152 of the present embodiment generates the second prompt 502 similar to the first prompt 501 on the basis of the first prompt 501 input by a user.

For example, when, as in the example illustrated in FIG. 2, the first prompt 501 includes an instruction to specify a specific type of cross section, such as "axial cross section", the prompt acquisition function 152 generates the second prompt 502 by replacing the type of cross section with another type of cross section. Regardless of the type of cross section, the prompt acquisition function 152 may generate the second prompt 502 by replacing text data wording included in the first prompt 501 with another wording that is medically synonymous.

The prompt acquisition function 152 may replace part of the first prompt 501 on the basis of a prescribed replacement rule. The prescribed replacement rule may be, for example, data indicating a correspondence between a wording to be replaced and a replacement wording. Such data may be stored in the storage circuitry 120. When the first prompt 501 includes a wording to be replaced (for example, "axial cross section"), the prompt acquisition function 152 replaces this wording with a replacement wording (for example, "sagittal cross section", "coronal cross section", etc.) defined in the rule.

Alternatively, the prompt acquisition function 152 may acquire the second prompt 502, in which a part of the first prompt 501 is replaced with another wording, by inputting the first prompt 501 into another generative model different from the generative model 40 to be evaluated. Other generative models may be, for example, LLMs that can process text data. Other generative models may be stored in the storage circuitry 120 of the first information processing apparatus 100, the second information processing apparatus 200, or other information processing apparatuses.

Except for the process of generating the second prompt 502 by the prompt acquisition function 152, the processes performed by the first information processing apparatus 100 of the present embodiment are the same as in the first embodiment.

In this way, by automatically generating the second prompt 502 similar to the first prompt 501, the first information processing apparatus 100 of the present embodiment can reduce the time and effort required for a user to generate the second prompt 502 for an evaluation process. In addition, by automatically generating the second prompt 502 in the first information processing apparatus 100, the occurrence of unintended errors caused by manual generation of the second prompt 502 by a user can be reduced, and also the occurrence of differences between the second prompt 502 and the first prompt 501 beyond the range of similarity can be reduced.

The prompt acquisition function 152 does not need to automatically generate all of the second prompt 502, and may have a function of checking the description of the second prompt 502 input by a user (review function), a function of making recommendations when the second prompt 502 is input, or the like.

For example, the prompt acquisition function 152 may determine whether the second prompt 502 input by a user differs from the first prompt 501 beyond the range of similarity. The fact that the second prompt 502 differs from the first prompt 501 beyond the range of similarity means that the second prompt 502 is not medically synonymous with the first prompt 501. For example, when the first prompt 501 has an instruction to output "information on a lesion" and the second prompt 502 has an instruction to output "information on a blood clot", the first prompt 501 and the second prompt 502 are not medically synonymous. When the second prompt 502 differs from the first prompt 501 beyond the range of similarity, since the first generated information 401 and the second generated information 402 output by the generative model 40 are not naturally similar, such a second prompt 502 is not suitable for use for evaluation purposes. Therefore, when the prompt acquisition function 152 determines that the second prompt 502 differs from the first prompt 501 beyond the range of similarity, the display control function 155 may control the display 140 to display the determination result.

The prompt acquisition function 152 may also generate candidates for the second prompt 502 before the second prompt 502 is input by a user. In this case, the display control function 155 may control the display 140 to display the candidates for the second prompt 502 generated by the prompt acquisition function 152 as recommendations.

### Third Embodiment

In the first embodiment described above, when the relevance between the first generated information 401 and the second generated information 402 is below the criterion, the first information processing apparatus 100 regenerates the first generated information 401 and repeats the evaluation. However, in the third embodiment, when the relevance between the first generated information 401 and the second generated information 402 is below the criterion, the first information processing apparatus 100 further generates a new prompt and performs the evaluation again on the basis of the new prompt.

The information processing system S of the present embodiment includes, for example, the first information processing apparatus 100, the second information processing apparatus 200, and the medical information system 300, as in the first embodiment described with reference to FIG. 1. The first information processing apparatus 100 of the present embodiment includes the NW interface 110, the storage circuitry 120, the input interface 130, the display 140, and the processing circuitry 150, as in the first embodiment.

The processing circuitry 150 of the present embodiment includes the reception function 151, the prompt acquisition function 152, the generated information acquisition function 153, the evaluation function 154, and the display control function 155, as in the first embodiment.

The reception function 151, the generated information acquisition function 153, and the display control function 155 have the same functions as in the first embodiment.

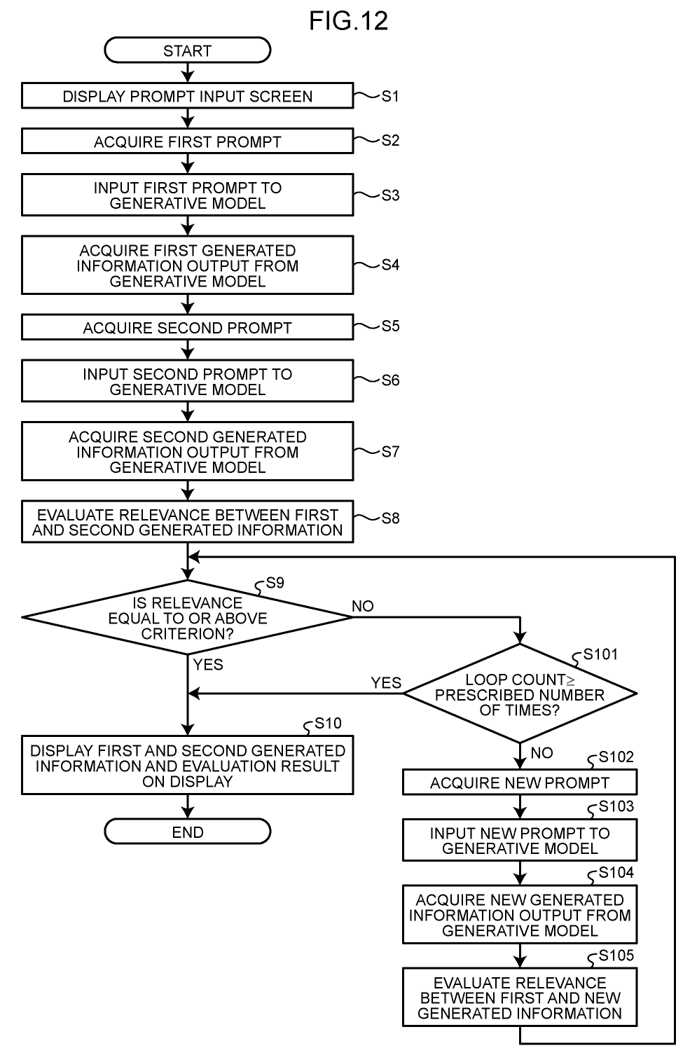

FIG. 12 is a flowchart showing an example of the procedure of the evaluation process according to the third embodiment. The process from the display of the prompt input screen at S1 to the determination of whether the relevance is equal to or above the criterion at S9 and the display of the evaluation results at S10 in FIG. 12 are the same as in the first embodiment.

If the relevance in the evaluation results is below the criterion, the evaluation function 154 of the present embodiment repeatedly evaluates the first generated information 401 with new generated information a prescribed number of times. The prescribed number of times in the loop in FIG. 12 is, for example, 10, but is not limited thereto.

The prompt acquisition function 152 of the present embodiment acquires a new prompt (S102) while the loop count has not reached the prescribed number of times ("No" at S101). In the method of acquiring the new prompt, the new prompt may be automatically generated by the prompt acquisition function 152, for example, similar to the method of generating the second prompt 502 in the second embodiment described above. Alternatively, the display control function 155 may control the display 140 to display an instruction for a user to input a new prompt, and the prompt acquisition function 152 may acquire the new prompt input by the user.

The new prompt is, for example, a third prompt, and is similar to the first prompt 501. More specifically, the new prompt is medically synonymous with the first prompt 501. The new prompt is a prompt different from other prompts (for example, the second prompt) generated prior to the new prompt. As a specific example, when the first and second prompts 501 and 502 include "axial cross section," "sagittal cross section," and "coronal cross section," the prompt acquisition function 152 generates the third prompt specifying a cross section in a direction different from the cutting direction of the three cross sections (for example, "cross section inclined at n° in the Z-axis direction of a subject", or the like).

Subsequently, the generated information acquisition function 153 inputs the new prompt acquired at S102 to the generative model 40 (S103).

Subsequently, the generated information acquisition function 153 acquires new generated information output from the generative model 40 (S104).

Subsequently, the evaluation function 154 evaluates the relevance between the first generated information 401 and the new generated information generated at S104 (S105). If the relevance is not equal to or above the criterion, the processes at S9 and S101 to S105 are repeated until the loop count reaches a prescribed number of times. In this case, the prompt acquisition function 152 acquires a new prompt with each loop.

If the relevance is equal to or above the criterion in the evaluation process using the new generated information, the procedure proceeds to the process at S10. The evaluation function 154 may take into account the loop count in the evaluation until the relevance is equal to or above the criterion. For example, the evaluation function 154 may subtract the evaluation for the relevance the greater the loop count until the relevance is equal to or above the criterion.

If the loop count has reached the prescribed number of times ("Yes" at S101), the evaluation function 154 fixes the latest evaluation results. Subsequently, the procedure proceeds to the process at S10, and the display control function 155 controls the display 140 to display the results of the evaluation for the relevance between the first generated information 401 and the second generated information 402 by the evaluation function 154. The procedure of the flowchart ends.

In this way, when the relevance in the evaluation results is below the criterion, the first information processing apparatus 100 of the present embodiment acquires a new prompt and evaluates the relevance between new generated information based on the new prompt and the first generated information 401. This allows the first information processing apparatus 100 of the present embodiment to improve the accuracy of evaluation results by performing the evaluation multiple times by using various generated information.

### Fourth Embodiment

In the first to third embodiments described above, the first information processing apparatus 100 uses the first prompt 501 and the second prompt 502 similar to the first prompt 501 for evaluation of generated information; however, in the fourth embodiment, a second prompt 502 identical to the first prompt 501 is used for evaluation of generated information.

The information processing system S of the present embodiment includes, for example, the first information processing apparatus 100, the second information processing apparatus 200, and the medical information system 300, as in the first embodiment described with reference to FIG. 1. The first information processing apparatus 100 of the present embodiment includes the NW interface 110, the storage circuitry 120, the input interface 130, the display 140, and the processing circuitry 150, as in the first embodiment.

The processing circuitry 150 of the present embodiment includes the reception function 151, the prompt acquisition function 152, the generated information acquisition function 153, the evaluation function 154, and the display control function 155, as in the first embodiment.

The reception function 151, the generated information acquisition function 153, the evaluation function 154, and the display control function 155 have the same functions as in the first embodiment.

The prompt acquisition function 152 of the present embodiment acquires the first prompt 501 and the second prompt 502 identical to the first prompt 501. For example, the first prompt 501 is input by a user. The prompt acquisition function 152 acquires the second prompt 502 by duplicating the first prompt 501.

Except for the process of acquiring the second prompt 502 by duplication by the prompt acquisition function 152, the processes performed by the first information processing apparatus 100 of the present embodiment are the same as in the first embodiment.

In this way, the first information processing apparatus 100 of the present embodiment evaluates the relevance between the first generated information 401 obtained by inputting the first prompt 501 to the generative model 40 and the second generated information 402 obtained by inputting the second prompt 502 identical to the first prompt 501 to the generative model 40, and outputs evaluation results. That is, in addition to the same effects as the first embodiment, the first information processing apparatus 100 of the present embodiment can verify fluctuations in the output of the generative model 40 when the same prompt is input.

### Fifth Embodiment

In the first to fourth embodiments described above, the medical image data 301 included in the first prompt 501 and the medical image data 301 included in the second prompt 502 are identical in the first information processing apparatus 100. In the fifth embodiment, the first prompt 501 and the second prompt 502 include three-dimensional first medical image data configured by different stack images.

The information processing system S of the present embodiment includes, for example, the first information processing apparatus 100, the second information processing apparatus 200, and the medical information system 300, as in the first embodiment described with reference to FIG. 1. The first information processing apparatus 100 of the present embodiment includes the NW interface 110, the storage circuitry 120, the input interface 130, the display 140, and the processing circuitry 150, as in the first embodiment.

The processing circuitry 150 of the present embodiment includes the reception function 151, the prompt acquisition function 152, the generated information acquisition function 153, the evaluation function 154, and the display control function 155, as in the first embodiment.

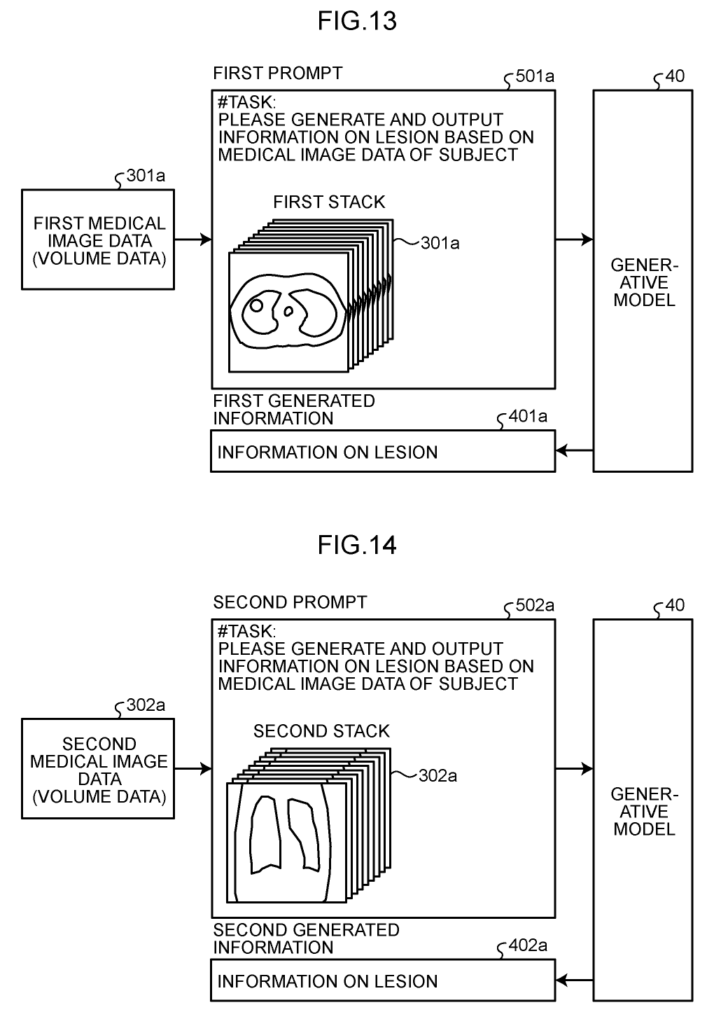

FIG. 13 is a diagram illustrating an example of a first prompt 501a and first generated information 401a according to the fifth embodiment. As illustrated in FIG. 13, the first prompt 501a includes three-dimensional first medical image data 301a configured by pieces of first stacked image data. The pieces of first stacked image data are, for example, pieces of two-dimensional image data of axial cross sections. The axial cross section is an example of a cross section in the first direction.

The first prompt 501a includes, for example, an instruction to cause the generative model 40 to generate, as the first generated information 401a, information on a first abnormal area (lesion) from the first medical image data 301a, such as "Please generate and output information on a lesion based on medical image data of a subject".

FIG. 14 is a diagram illustrating an example of a second prompt 502a and second generated information 402a according to the fifth embodiment. As illustrated in FIG. 14, the second prompt 502a includes three-dimensional second medical image data 302a configured by pieces of second stacked image data. The pieces of second stacked image data are pieces of two-dimensional image data of cross sections, whose type is different from that of the pieces of first stacked image data. In one example, the pieces of second stacked image data are pieces of two-dimensional image data of coronal cross sections. The coronal cross section is an example of a cross section in the second direction.

A three-dimensional object captured in the first medical image data 301a and a three-dimensional object captured in the second medical image data 302a are the same. That is, the first medical image data 301a and the second medical image data 302a are medically synonymous image data, except that cross sectional directions of the stacked image data constituting the image data are different.

As illustrated in FIG. 14, the second prompt 502a includes, for example, the same text data as the first prompt 501a such as "Please generate and output information on a lesion based on medical image data of a subject". Therefore, the first prompt 501a and the second prompt 502a are substantially synonymous medically.

The procedure of an evaluation process in the present embodiment is the same as in the first embodiment described with reference to FIG. 11.

In this way, the first information processing apparatus 100 of the present embodiment evaluates the relevance between the first generated information 401a obtained by inputting, to the generative model 40, the first prompt 501a including the three-dimensional first medical image data 301a configured by pieces of the first stacked image data and the second generated information 402a obtained by inputting, to the generative model 40, the second prompt 502a including the three-dimensional second medical image data 302a configured by pieces of the second stacked image data different from the pieces of first stacked image data, and outputs evaluation results. In addition to the same effects as the first embodiment, the first information processing apparatus 100 of the present embodiment can verify fluctuations in the first and second generated information 401a and 402a due to differences in the type of stacked image data.

### Sixth Embodiment

In the sixth embodiment, image conditions of medical image data included in first and second prompts are different.

The information processing system S of the present embodiment includes, for example, the first information processing apparatus 100, the second information processing apparatus 200, and the medical information system 300, as in the first embodiment described with reference to FIG. 1. The first information processing apparatus 100 of the present embodiment includes the NW interface 110, the storage circuitry 120, the input interface 130, the display 140, and the processing circuitry 150, as in the first embodiment.

The processing circuitry 150 of the present embodiment includes the reception function 151, the prompt acquisition function 152, the generated information acquisition function 153, the evaluation function 154, and the display control function 155, as in the first embodiment.

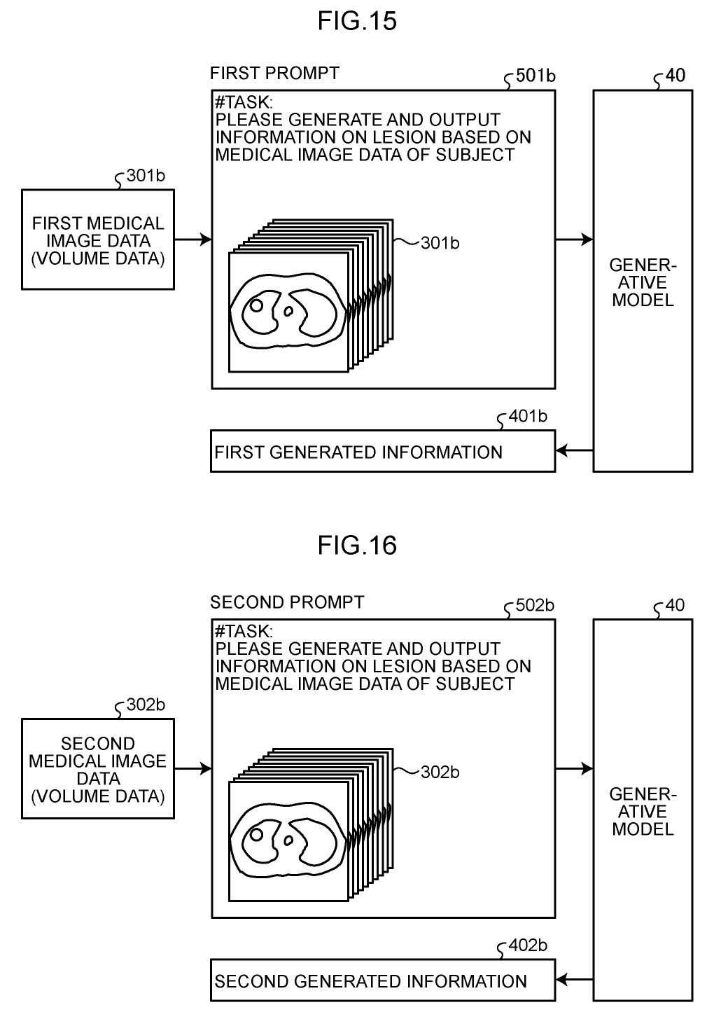

FIG. 15 is a diagram illustrating an example of a first prompt 501b and first generated information 401b according to the sixth embodiment. As illustrated in FIG. 15, the first prompt 501b includes first medical image data 301b generated under a first image condition. The first prompt 501b also includes text data instructing the generative model 40 to generate and output first generated information based on the first medical image data 301b, such as "Please generate and output information on a lesion based on medical image data of a subject". When the first medical image data 301b is distinguished from the first medical image data in other embodiments, the first medical image data 301b may be referred to as third medical image data.

FIG. 16 is a diagram illustrating an example of a second prompt 502b and second generated information 402b according to the sixth embodiment. As illustrated in FIG. 16, the second prompt 502b includes second medical image data 302b generated under a second image condition. The second prompt 502b includes the same text data as the first prompt 501b. When the second medical image data 302b is distinguished from the second medical image data in other embodiments, the second medical image data 302b may be referred to as fourth medical image data.

The second image condition is an image condition different from the first image condition. The first image condition and the second image condition include at least one of an imaging condition or an image processing condition. Specifically, the first image condition and the second image condition include, for example, reconstruction conditions, back projection, and conditions related to successive approximation. The first image condition and the second image condition include, for example, contrast and non-contrast conditions for imaging. The first image condition and the second image condition may define, for example, a sequence during which magnetic resonance image data is taken.

Specifically, when the first medical image data 301b and the second medical image data 302b are CT image data, the first image condition may be "contrast; slice thickness 5 mm; kernel FC 51; phase 30%" and the second image condition may be "non-contrast; slice thickness 0.5 mm; kernel FC 30; phase 70%". The examples are given for illustrative purposes only, and the first image condition and the second image condition are not limited to such examples.

The first medical image data 301b and the second medical image data 302b are image data obtained by imaging the same subject in the same imaging range. That is, the first medical image data 301b and the second medical image data 302b are assumed to be identical except for the image conditions at the time of generation.

The text data included in the first and second prompts 501b and 502b are not limited to the examples illustrated in FIGS. 15 and 16. For example, the text data may instruct the generated information to include at least one of an abnormal area (including a lesion), a similar image, or a diagnosis name. In this case, the first generated information 401b and the second generated information 402b in the present embodiment include at least one of abnormal areas in the first medical image data 301b and the second medical image data 302b, similar image data for the first medical image data 301b and the second medical image data 302b, or a diagnosis name based on the first medical image data 301b and the second medical image data 302b.

The procedure of an evaluation process in the present embodiment is the same as in the first embodiment described in FIG. 11.

In this way, the first information processing apparatus 100 of the present embodiment evaluates the relevance between the first generated information 401b obtained by inputting, to the generative model 40, the first prompt 501b including the first medical image data 301b generated under the first image condition and the second generated information 402b obtained by inputting, to the generative model 40, the second prompt 502b including the second medical image data 302b generated under the second image condition different from the first image condition, and outputs evaluation results. Therefore, in addition to the same effects as the first embodiment, the first information processing apparatus 100 of the present embodiment can verity fluctuations in the output of the generative model 40 due to differences in image conditions of the first and second medical image data 301b and 302b included in the first and second prompts 501b and 502b.

For example, when training data in prior training of the generative model 40 is biased toward image data generated under specific image conditions, the accuracy of generated information for prompts including image data generated under conditions other than the specific image conditions may be low. In such a case, the first information processing apparatus 100 of the present embodiment can be applied to evaluate the bias in the accuracy of the generative model 40.

The content and subject matter of the image conditions are not limited to the above examples. For example, the image conditions may be imposed on the medical image data generated by the generative model 40 on the basis of the first and second prompts 501b and 502b.

For example, the first prompt 501b is assumed to include an instruction to generate ES (systolic) phase image data from three-dimensional medical image data 301 obtained by imaging the chest of a subject for one cardiac cycle, and output the generated image data. The second prompt 502b is assumed to include an instruction to generate ED (diastolic) phase image data from three-dimensional medical image data 301 obtained by imaging the chest of the subject for one cardiac cycle, and output the generated image data. In this case, the ES phase and the ED phase are examples of the image conditions. In this case, since only the difference between the first generated information 401b and the second generated information 402b is the phase, when fluctuations in the generative model 40 are small, the image data included in the first generated information 401b and the image data included in the second generated information 402b are identical or very similar in areas other than the heart. For example, the evaluation function 154 may distinguish the heart or lungs, whose shape changes significantly with phase, as regions of interest, and other organs or bones, whose shape changes less with phase, as regions of non-interest. The evaluation function 154 may evaluate that the higher the relevance between the first generated information 401b and the second generated information 402b, the higher the degree of similarity between regions of non-interest in the image data included in the first generated information 401b and the image data included in the second generated information 402b.

The first prompt 501b may include an instruction to generate and output an image of the ED phase on the basis of medical image data of the ES phase. In this case, the corresponding second prompt 502b may include an instruction to generate and output an image of the ES phase on the basis of medical image data of the ED phase. In this way, the evaluation function 154 may compare the first generated information 401b and the second generated information 402b generated on the basis of the first and second prompts 501b and 502b with other aligned conditions with each other by using the difference in image conditions as a phase difference.

### Seventh Embodiment

In the first to sixth embodiments described above, the first prompts 501, 501a, and 501b and the second prompts 502, 502a, and 502b include medical image data. However, each prompt may include no medical image data. In the seventh embodiment, the first and second prompts include no medical image data, and include medical information in the form of text data.

The information processing system S of the present embodiment includes, for example, the first information processing apparatus 100, the second information processing apparatus 200, and the medical information system 300, as in the first embodiment described with reference to FIG. 1. The first information processing apparatus 100 of the present embodiment includes the NW interface 110, the storage circuitry 120, the input interface 130, the display 140, and the processing circuitry 150, as in the first embodiment.

The processing circuitry 150 of the present embodiment includes the reception function 151, the prompt acquisition function 152, the generated information acquisition function 153, the evaluation function 154, and the display control function 155, as in the first embodiment. The reception function 151 and the display control function 155 have the same functions as the first embodiment.

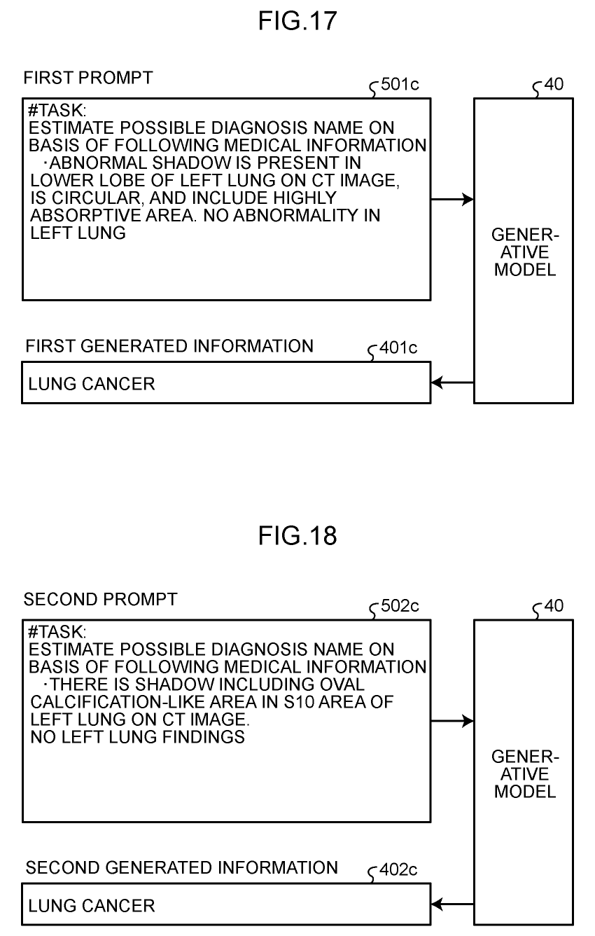

FIG. 17 is a diagram illustrating an example of a first prompt 501c and first generated information 401c according to the seventh embodiment. As illustrated in FIG. 17, the first prompt 501c includes no image data. The first prompt 501c includes text data indicating an instruction to the generative model 40, such as "Estimate a possible diagnosis name on the basis of the following medical information" and medical information in the form of text data such as "An abnormal shadow is present in the lower lobe of the left lung on the CT image, is circular, and includes a highly absorptive area. No abnormality in the left lung". The medical information is acquired, for example, from the medical information system 300. Alternatively, the medical information may be directly written by a user in the body of the first prompt. The medical information included in the first prompt 501c is an example of first medical information.

In the example illustrated in FIG. 17, the generative model 40 receives the first prompt 501c and outputs a diagnosis name "lung cancer" as the first generated information 401c.

FIG. 18 is a diagram illustrating an example of a second prompt 502c and second generated information 402c according to the seventh embodiment. As in the first prompt 501c, the second prompt 502c includes no image data. The second prompt 502c includes text data of the same instruction as the instruction included in the first prompt 501c, such as "Estimate a possible diagnosis name on the basis of the following medical information".

The second prompt 502c also includes medical information in the form of text data such as "There is a shadow including an oval calcification-like area in the S10 area of the left lung on the CT image. No left lung findings". The medical information is the first medical information included in the first prompt 501c with some of the wording replaced with other wording similar to the wording. The medical information included in the second prompt 502c is an example of second medical information.

Similarity in wording between the first medical information and the second medical information refers to, for example, a case in which wording before replacement is a subordinate or superordinate concept of wording after replacement, a case in which the relationship between the wording before replacement and the wording after replacement is an expression representing a degree (high/low, big/small) or a correspondence between an adjective and a numerical value, or the like. The first medical information and the second medical information differ in expression, but are assumed to be medically synonymous.

For example, in the examples illustrated in FIGS. 17 and 18, wordings such as "lower lobe of left lung", "circular", and "highly absorptive area" in the first medical information included in the first prompt 501c are replaced with the more specific subordinate wordings such as "S10 area of left lung", "oval", and "calcification-like area" in the second medical information included in the second prompt 502c.

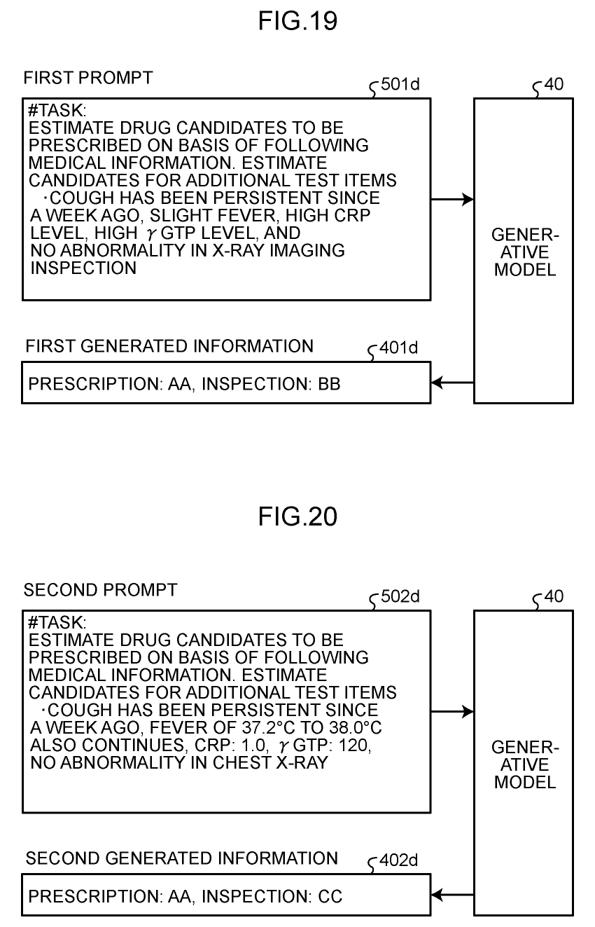

FIG. 19 is a diagram illustrating another example of a first prompt 501d and first generated information 401d according to the seventh embodiment. In the example illustrated in FIG. 19, the first prompt 501d includes text data indicating an instruction to the generative model 40 such as "Estimate drug candidates to be prescribed on the basis of the following medical information. Estimate candidates for additional test items", and first medical information in the form of text data such as "Cough has been persistent since a week ago, slight fever, high CRP level, high γGTP level, and no abnormality in X-ray imaging inspection".

FIG. 20 is a diagram illustrating another example of a second prompt 502d and second generated information 402d according to the seventh embodiment. The second prompt 502d illustrated in FIG. 20 is similar to the first prompt 501d illustrated in FIG. 19. Specifically, the second prompt 502d illustrated in FIG. 20 includes text data indicating the same instruction to the generative model 40 as the first prompt 501d, and second medical information in the form of text data such as "Cough has been persistent since a week ago, fever of 37.2°C to 38.0°C also continues, CRP: 1.0:, γGTP: 120, no abnormality in chest x-ray". The second medical information illustrated in FIG. 20 replaces the expressions representing a degree in the first medical information illustrated in FIG. 19 with specific numerical values.

The prompt acquisition function 152 of the present embodiment acquires the first prompts 501c and 501d and the second prompts 502c and 502d. The method of acquiring the first prompts 501c and 501d and the second prompts 502c and 502d may be input by a user as in the first embodiment. Alternatively, as in the second embodiment, only the first prompts 501c and 501d may be generated by input by a user, and the prompt acquisition function 152 may generate the second prompts 502c and 502d on the basis of the first prompts 501c and 501d.

When both the first prompts 501c and 501d and the second prompts 502c and 502d are generated by input by a user, the prompt acquisition function 152 may generate a replacement target part and candidates for replacement wordings from the wordings of the first medical information included in the first prompts 501c and 501d. In this case, the display control function 155 may suggest the replacement to the user by controlling the display 140 to display the replacement target part and the candidates for replacement wordings.

As in the first embodiment, the generated information acquisition function 153 of the present embodiment inputs the first prompts 501c and 501d and the second prompts 502c and 502d acquired by the prompt acquisition function 152 to the generative model 40 of the second information processing apparatus 200 via the network 400 and the NW interface 110, and acquires the first generated information 401c and 401d and the second generated information 402c and 402d from the generative model 40.

Since contents of the first prompts 501c and 501d and the second prompts 502c and 502d are medically synonymous, the first generated information 401c and 401d and the second generated information 402c and 402d are identical or similar when the generative model 40 has been properly trained. In the example illustrated in FIG. 18, the second generated information 402c is the same diagnosis name "lung cancer" as the first generated information 401c.

However, differences occur between the first generated information 401c and 401d and the second generated information 402c and 402d due to superficial wording differences between the first prompts 501c and 501d and the second prompts 502c and 502d, such as when there are biases in the prior training of the generative model 40. In the examples illustrated in FIGS. 19 and 20, the first generated information 401d is "Prescription: AA, Inspection: BB", while the second generated information 402d is "Prescription: AA, Inspection: CC", resulting in some differences.

The evaluation function 154 of the present embodiment, for example, evaluates the degree of the relevance between the first generated information 401c and 401d and the second generated information 402c and 402d by the degree of similarity between the first generated information 401c and 401d and the second generated information 402c and 402d, as in the first embodiment. In other words, the higher the degree of similarity between the first generated information 401c and 401d and the second generated information 402c and 402d, the evaluation function 154 evaluates that the reliability of the generative model 40 is higher.

For example, in a case in which the first prompts 501c and 501d and the second prompts 502c and 502d include instructions to output diagnosis names, prescriptions, or inspection items as illustrated in FIGS. 17 to 20, when the diagnosis name included in the first generated information 401c and 401d and the diagnosis name included in the second generated information 402c and 402d are identical or similar, the evaluation function 154 may evaluate that the relevance between the first generated information 401c and 401d and the second generated information 402c and 402d is high. When the first generated information 401c and 401d and the second generated information 402c and 402d each include a plurality of diagnosis names, the evaluation function 154 may evaluate that the greater the number of identical diagnosis names in the first generated information 401c and 401d and the second generated information 402c and 402d, the higher the relevance between the first generated information 401c and 401d and the second generated information 402c and 402d.

In this way, in addition to the same effects as in the first embodiment, even when the first prompts 501c and 501d and the second prompts 502c and 502d include medical information in the form of text data, the first information processing apparatus 100 of the present embodiment can evaluate the relevance between the first generated information 401c and 401d and the second generated information 402c and 402d.

### Eighth Embodiment

In the first to seventh embodiments described above, the first information processing apparatus 100, which is a computer such as a server or a PC, is an example of an information processing apparatus. In the eighth embodiment, an X-ray CT apparatus performs a process as an example of the information processing apparatus.

The information processing system S of the present embodiment includes, as an example, an X-ray CT apparatus, the second information processing apparatus 200, and the medical information system 300. The second information processing apparatus 200 and the medical information system 300 have the same functions as the first embodiment.

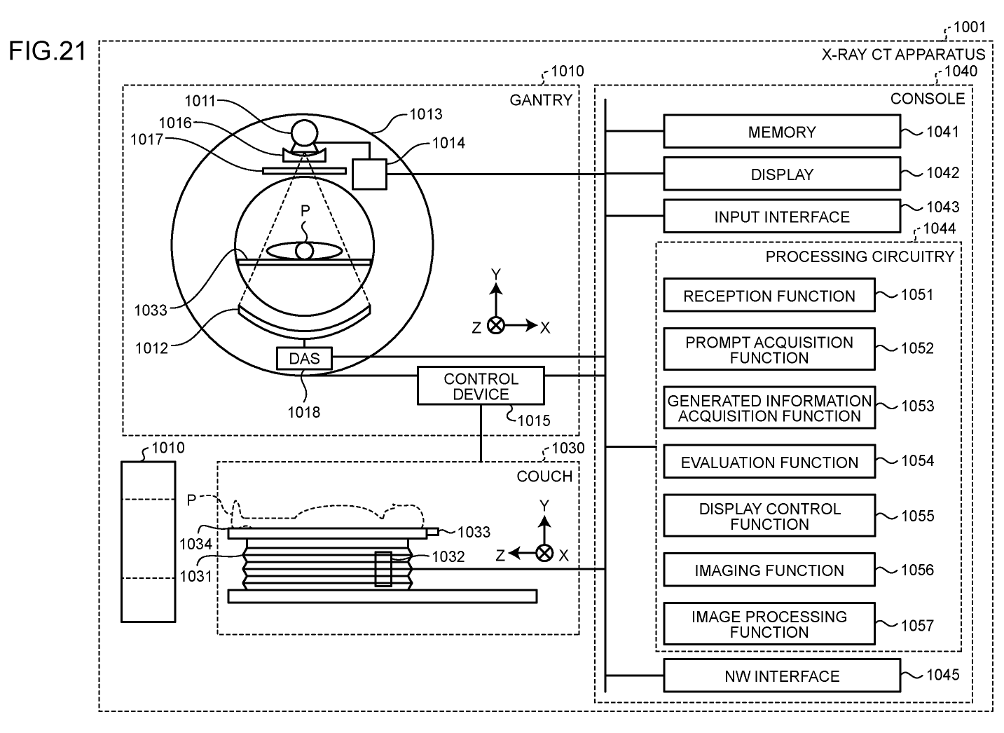

FIG. 21 is a diagram illustrating an example of a hardware configuration of an X-ray CT apparatus 1001 according to the eighth embodiment. The X-ray CT apparatus 1001 emits X-rays from an X-ray tube 1011 to a subject P, and detects the emitted X-rays by an X-ray detector 1012. The X-ray CT apparatus 1001 generates CT images related to the subject P on the basis of output from the X-ray detector 1012.

As illustrated in FIG. 21, the X-ray CT apparatus 1001 has a gantry 1010, a couch 1030, and a console 1040. FIG. 21 illustrates a plurality of the gantries 1010 for illustrative purposes. The gantry 1010 is a scanning device with a configuration for X-ray CT imaging of the subject P. The couch 1030 is a transport device for placing and positioning the subject P that is a target of X-ray CT imaging. The console 1040 is a computer that controls the gantry 1010.

As illustrated in FIG. 21, the gantry 1010 includes the X-ray tube 1011, the X-ray detector 1012, a rotating frame 1013, an X-ray high-voltage device 1014, a control device 1015, a wedge 1016, a collimator 1017, and a data acquisition system (DAS) 1018. In the present embodiment, the rotation axis of the rotating frame 1013 or the longitudinal direction of a couch top 1033 of the couch 1030 in a non-tilted state is defined as a Z-axis direction, the axis direction orthogonal to the Z-axis direction and horizontal to a floor surface is defined as an X-axis direction, and the axis direction orthogonal to the Z-axis direction and vertical to the floor surface is defined as a Y-axis direction.

The X-ray tube 1011 irradiates the subject P with X-rays by emitting thermoelectrons from a cathode to an anode by using a high voltage supplied by the X-ray high-voltage device 1014.

The X-ray detector 1012 detects X-rays emitted from the X-ray tube 1011 and passing through the subject P. The X-ray detector 1012 outputs electrical signals corresponding to the detected X-ray dose to the DAS 1018. The X-ray detector 1012 is, for example, an indirect conversion detector with a grid, a scintillator array, and an optical sensor array. The scintillator array has a plurality of scintillators.

The rotating frame 1013 is an annular frame that supports the X-ray tube 1011 and the X-ray detector 1012 opposite each other and rotates the X-ray tube 1011 and the X-ray detector 1012 by the control device 1015 to be described below. In addition, the rotating frame 1013 can further support various configurations not illustrated in FIG. 21.

The X-ray high-voltage device 1014 has a high-voltage generator and an X-ray controller. The high-voltage generator has electrical circuitry such as a transformer and a rectifier, and generates the high voltage to be applied to the X-ray tube 1011 and a filament current to be supplied to the X-ray tube 1011. The X-ray controller controls an output voltage according to the X-rays emitted by the X-ray tube 1011.

The control device 1015 includes a drive mechanism such as a motor and an actuator, and processing circuitry having a processor that controls the drive mechanism, a memory, and the like. The control device 1015 receives input signals from an input interface 1043, other input interfaces provided on the gantry 1010, and the like, and controls the operation of the gantry 1010 and the couch 1030. The control device 1015 may be provided on the gantry 1010 or on the console 1040.

The wedge 1016 is a filter for adjusting the X-ray dose emitted from the X-ray tube 1011.

The collimator 1017 slidably supports a plurality of lead plates serving to shield X-rays, and limits the irradiation range of X-rays transmitted through the wedge 1016 by adjusting the shape of a slit formed by the lead plates.

The DAS 1018 reads electrical signals corresponding to the X-ray dose detected by the X-ray detector 1012 from the X-ray detector 1012. The DAS 1018 amplifies the read electrical signals and integrates (adds) the electrical signals over a view period to collect detection data with digital values corresponding to the X-ray dose over the view period. The detection data is referred to as projection data. The DAS 1018 is implemented, for example, by an application specific integrated circuit (ASIC) provided with circuit elements capable of generating projection data. The projection data is transmitted to the console 1040 via a non-contact data transmission device or the like.

The detection data generated by the DAS 1018 is transmitted to the console 1040. The transmission method of the detection data is, for example, optical communication, but any non-contact data transmission method can be used.

In the present embodiment, the X-ray CT apparatus 1001 provided with the integrating type X-ray detector 1012 is described as an example; however, the technology according to the present embodiment can also be implemented as an X-ray CT apparatus 1001 provided with a photon-counting type X-ray detector.

The couch 1030 is a device for placing and moving the subject P to be scanned. The couch 1030 has a base 1031, a couch drive unit 1032, the couch top 1033, and a support frame 1034.

The console 1040 has a memory 1041, a display 1042, the input interface 1043, processing circuitry 1044, and a NW interface 1045. Data communication among the memory 1041, the display 1042, the input interface 1043, the NW interface 1045, and the processing circuitry 1044 is performed via a bus (BUS). The console 1040 is described as a separate unit from the gantry 1010, but the gantry 1010 may include the console 1040 or some of the components of the console 1040.

The memory 1041 is implemented, for example, by a semiconductor memory element such as a ROM, a RAM, and a flash memory, a hard disk, an optical disk, or the like. For example, the memory 1041 stores various data to be stored. The memory 1041 stores projection data and reconstructed image data. For example, the memory 1041 stores various computer programs. The storage area of the memory 1041 may be in the X-ray CT apparatus 1001 or in an external storage device connected via a network.

The display 1042 is a liquid crystal display, an organic EL display, a CRT display, or the like for displaying various information.

The input interface 1043 receives various input operations from an operator, converts the received input operations into electrical signals, and outputs the electrical signals to the processing circuitry 1044.

As the input interface 1043, for example, a mouse, a keyboard, a trackball, a switch, a button, a joystick, a touchpad, a touch panel display, or the like can be used as appropriate. In the present embodiment, the input interface 1043 is not limited to those with these physical operating components. For example, electrical signal processing circuitry that receives electrical signals corresponding to input operations from an external input device provided separately from the device and outputs the electrical signals to the processing circuitry 1044 is also included in an example of the input interface 1043.

The NW interface 1045 is connected to the processing circuitry 1044 and controls the transmission and communication of various data performed between the X-ray CT apparatus 1001 and the second information processing apparatus 200/the medical information system 300. The NW interface 1045 is implemented by a network card, a network adapter, a NIC, or the like.

The processing circuitry 1044 controls the operation of the entire X-ray CT apparatus 1001. The processing circuitry 1044 has a processor and a memory such as a ROM and a RAM as hardware resources. The processing circuitry 1044 performs various system processes by a processor that executes a computer program loaded into the memory. The processing circuitry 1044 includes, for example, a reception function 1051, a prompt acquisition function 1052, a generated information acquisition function 1053, an evaluation function 1054, a display control function 1055, an imaging function 1056, and an image processing function 1057.

The reception function 1051, the prompt acquisition function 1052, the generated information acquisition function 1053, the evaluation function 1054, the display control function 1055 of the present embodiment have the same functions as the reception function 151, the prompt acquisition function 152, the generated information acquisition function 153, the evaluation function 154, and the display control function 155 in the processing circuitry 150 of the first embodiment.

The imaging function 1056 and the image processing function 1057 are functions for capturing X-ray CT images.

For example, the imaging function 1056 controls CT scans performed on the gantry 1010 to obtain the detection data of the X-rays detected by the X-ray detector 1012. The imaging function 1056 performs a reconstruction process on the detection data of the X-rays to obtain X-ray CT image data obtained by capturing the subject P. Examples of the reconstruction process include a filter back-projection method, a successive approximation reconstruction method, and machine learning. The image processing function 1057 converts the X-ray CT image data into tomographic image data or three-dimensional image data of a cross section by known methods on the basis of input operations received from the operator via the input interface 1043.

The medical image data included in the first and second prompts to be input to the generative model 40 of the present embodiment is, for example, X-ray CT image data captured by the imaging function 1056 and the image processing function 1057.

In the present embodiment, the X-ray CT apparatus 1001 is an example of the information processing apparatus; however, another medical image diagnostic apparatus may be an example of the information processing apparatus. For example, various medical image diagnostic apparatuses, such as an X-ray diagnostic apparatus, a magnetic resonance imaging (MRI) apparatus, an ultrasound diagnostic apparatus, a single photon emission computed tomography (SPECT) apparatus, a positron emission computed tomography (PET) apparatus, a SPECT-CT apparatus that integrates the SPECT apparatus and the X-ray CT apparatus, and a PET-CT apparatus that integrates the PET apparatus and the X-ray CT apparatus, may have the functions of the processing circuitry 1044 illustrated in FIG. 21.

### First Modification

In each of the above-described embodiments, the information processing system S is assumed to be provided in a medical institution such as a hospital; however, the information processing system S may be provided in a company or the like other than a medical institution. In addition, although the above-described embodiments describe examples of using the information processing system S in the medical field, the field of use of the information processing system S is not limited to the medical field and can be used in various businesses, research and development, and the like that utilize generative AI. When the information processing system S is used in fields other than the medical field, the information processing system S does not need to include the medical information system 300. For example, the information processing system S may include the first information processing apparatus 100 and the generative model 40. The information processing system S may include other database management apparatuses or the like, instead of or in addition to the medical information system 300.

### Second Modification

In each of the above-described embodiments, an example is described in which the first information processing apparatus 100, the second information processing apparatus 200, and the medical information system 300 are connected via the network 400 such as an in-hospital LAN, as illustrated in FIG. 1 and the like; however, the configuration of the information processing system S is not limited thereto. For example, the second information processing apparatus 200 may be provided outside a medical institution. As a specific example, the second information processing apparatus 200 may be a server or the like of a service provider that provides generative AI as a service. In this case, the second information processing apparatus 200 may be connected to the first information processing apparatus 100 and the medical information system 300 via the Internet or the like instead of an in-hospital LAN.

### Third Modification

The generative model 40 may be provided in the first information processing apparatus 100 instead of the second information processing apparatus 200. For example, the generative model 40 may be stored in the storage circuitry 120 of the first information processing apparatus 100 and used by the generated information acquisition function 153 described above. Alternatively, the generated information acquisition function 153 may include the generative model 40.

### Fourth Modification

In each of the above-described embodiments, the generative model 40 is assumed to be a multimodal model; however, the technology for constructing the generative model 40 is not limited thereto and various AI models can be employed.

### Fifth Modification

In each of the above-described embodiments, the first information processing apparatus 100 is assumed to output evaluation results by display on the display 140 or by other methods; however, the first information processing apparatus 100 does not necessarily have to output the evaluation results externally. For example, the term "output" may refer to the fact that the evaluation function 154 of the first information processing apparatus 100 stores the evaluation results in the storage circuitry 120 or the like.

Alternatively, the first information processing apparatus 100 may use the evaluation results only for internal processing. For example, when the evaluation result is below a prescribed criterion, the first information processing apparatus 100 may perform a process of deleting the first generated information 401. In this case, the evaluation results do not necessarily have to be output.

Various data handled in the present specification are typically digital data.

According to at least one embodiment described above, the reliability of the generated information generated by the generative model can be evaluated.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An X-ray CT apparatus comprising:
an X-ray tube configured to irradiate a subject with X-rays;
an X-ray detector configured to detect the X-rays emitted from the X-ray tube;
at least one memory; and
at least one piece of processing circuitry being connected to the memory and being configured to
acquire a first prompt including medical information and a second prompt similar to the first prompt,
input the first prompt to a generative model and acquire first generated information corresponding to the first prompt from the generative model,
input the second prompt into the generative model and acquire second generated information corresponding to the second prompt from the generative model, and
evaluate relevance between the first generated information and the second generated information.

2. An information processing apparatus comprising:
at least one memory; and
at least one piece of processing circuitry being connected to the memory and being configured to
acquire a first prompt including medical information and a second prompt similar to the first prompt,
input the first prompt to a generative model and acquire first generated information corresponding to the first prompt from the generative model,
input the second prompt into the generative model and acquire second generated information corresponding to the second prompt from the generative model, and
evaluate relevance between the first generated information and the second generated information.

3. The information processing apparatus according to claim 2, wherein the processing circuitry is configured to output results of the evaluation for the relevance between the first generated information and the second generated information.

4. The information processing apparatus according to claim 3, wherein the processing circuitry is configured to output the results of the evaluation for the relevance between the first generated information and the second generated information together with the first generated information and the second generated information.

5. The information processing apparatus according to claim 3, wherein
the first prompt and the second prompt each include at least one of medical image data or medical-related text data, and
the processing circuitry is configured to output the results of the evaluation for the relevance between the first generated information and the second generated information together with the medical image data, the first generated information in the medical image data, and the second generated information in the medical image data.

6. The information processing apparatus according to claim 2, wherein
the first prompt and the second prompt each include at least one of medical image data or medical-related text data,
the first prompt and the second prompt differ at least in part and are identical except for the difference, and
the processing circuitry is configured to evaluate a degree of the relevance between the first generated information and the second generated information by a degree of similarity between the first generated information and the second generated information.

7. The information processing apparatus according to claim 6, wherein
the first prompt includes an instruction to cause the generative model to generate, as the first generated information, information on a first abnormal area in a first cross section of the medical image data,
the second prompt includes an instruction to cause the generative model to generate, as the second generated information, information on a second abnormal area in a second cross section different from the first cross section of the medical image data, and
the processing circuitry is configured to evaluate the relevance between the first generated information and the second generated information on the basis of at least one of locations, area ranges, or number of the first abnormal area included in the first generated information and the second abnormal area included in the second generated information.

8. The information processing apparatus according to claim 2, wherein
the first prompt includes three-dimensional first medical image data configured by pieces of first stacked image data,
the second prompt includes three-dimensional second medical image data configured by pieces of second stacked image data different from the pieces of first stacked image data,
a three-dimensional object imaged in the first medical image data and a three-dimensional object imaged in the second medical image data are the same,
the pieces of first stacked image data are each two-dimensional image data in which the three-dimensional object is represented in a cross section in a first direction, and
the pieces of second stacked image data are each two-dimensional image data in which the three-dimensional object is represented in a cross section in a second direction different from the first direction.

9. The information processing apparatus according to claim 2, wherein
the first prompt includes an instruction to cause the generative model to generate the first generated information on the basis of third medical image data generated under a first image condition,
the second prompt includes an instruction to cause the generative model to generate the second generated information on the basis of fourth medical image data generated under a second image condition different from the first image condition, and
the first image condition and the second image condition include at least one of an imaging condition or an image processing condition.

10. The information processing apparatus according to claim 2, wherein
the first prompt includes first medical information in a form of text data,
the second prompt includes second medical information in a form of text data in which a part of a wording in the first medical information is replaced with another wording similar to the part of the wording, and
the processing circuitry evaluates a degree of the relevance between the first generated information and the second generated information by a degree of similarity between the first generated information and the second generated information.

11. An information processing method comprising:
acquiring a first prompt including medical information and a second prompt similar to the first prompt;
inputting the first prompt to a generative model and acquiring first generated information corresponding to the first prompt from the generative model;
inputting the second prompt into the generative model and acquiring second generated information corresponding to the second prompt from the generative model; and
evaluating relevance between the first generated information and the second generated information.

12. A non-transitory computer readable medium on which programmed instructions are recorded, the programmed instructions causing a computer to execute:
acquiring a first prompt including medical information and a second prompt similar to the first prompt;
inputting the first prompt to a generative model and acquiring first generated information corresponding to the first prompt from the generative model;
inputting the second prompt into the generative model and acquiring second generated information corresponding to the second prompt from the generative model; and
evaluating relevance between the first generated information and the second generated information.
